(19) United States
(12) Patent Application Publication
(10) Pub. No.: US 2025/0263692 A1
(43) Pub. Date: Aug. 21, 2025

(54) CURING FOR ITERATIVE NUCLEIC ACID-GUIDED NUCLEASE EDITING

(71) Applicant: Inscripta, Inc., Pleasanton, CA (US)

(72) Inventors: Andrew Garst, Pleasanton, CA (US); Andres Chaparro, Pleasanton, CA (US)

(21) Appl. No.: 18/856,423

(22) PCT Filed: Apr. 11, 2023
(86) PCT No.: PCT/US2023/018138

§ 371 Date: Oct. 11, 2024

Related U.S. Application Data
(60) Provisional application No. 63/330,260, filed on Apr. 12, 2022.

(51) Int. Cl. C12N 15/10

(52) U.S. Cl. CPC C12N 15/1082

(57) ABSTRACT

The present disclosure provides systems, methods, and compositions for performing iterative genomic editing of live cells with curing of editing vectors from prior rounds of editing.

### CROSS REFERENCE TO RELATED APPLICATIONS

This application claims the benefit of U.S. Provisional Application No. 63/330,260, filed Apr. 12, 2022, the content of which is incorporated herein by reference in its entirety.

### FIELD OF THE INVENTION

The present disclosure relates to systems, methods, and compositions for performing iterative genomic editing.

### BACKGROUND OF THE INVENTION

In the following discussion, certain articles and methods will be described for background and introductory purposes. Nothing contained herein is to be construed as an “admission” of prior art. Applicant expressly reserves the right to demonstrate, where appropriate, that the methods referenced herein do not constitute prior art under the applicable statutory provisions.

Iterative design and optimization are hallmarks of all engineering or product development processes, including those for biological systems. As in other disciplines, engineering cell strains requires multiple design-build-test-learn iterations, or rounds, to produce a desired production phenotype.

Generally, engineering cell strains for a desired production phenotype relies on the ability to make precise, targeted, and permanent changes to the cell genome over two to many rounds of editing. Recently, various CRISPR-based nucleic acid-guided cell editing methods have been developed, which show much promise in the realm of cell strain engineering. However, such methods lack the mechanisms for reliably and rapidly introducing new edits over multiple rounds, and are therefore typically limited to a small number of iterative editing operations (e.g., 2-3 rounds) targeting only a few pre-defined genomic loci.

Accordingly, there is a need in the art of nucleic acid-guided cell editing for improved systems, methods, and compositions for iterative editing. The present disclosure addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In one aspect, the disclosure provides, and includes, a method for iterative nucleic acid-guided nuclease editing, comprising: (i) providing a plurality of edited cells, the edited cells modified during an initial round of editing to include a first genomic edit, the edited cells comprising: a first editing vector comprising a first editing cassette and a first selectable marker; (ii) transforming the cells with a second editing vector, the second editing vector comprising: a second editing cassette, a second selectable marker, and a first nucleic acid sequence coding for a first curing gRNA configured to target the first selectable marker of the first editing vector; (iii) providing conditions for curing the first editing vector in the plurality of edited cells, wherein curing the first editing vector comprises cleaving the first editing vector at the first selectable marker with a nucleic acid-guided nuclease guided by the first curing gRNA; and (iv) providing conditions for editing the plurality of edited cells with the second editing cassette, wherein the second editing cassette is configured to further modify the edited cells to include a second genomic edit.

In one aspect, the disclosure provides, and includes, a method for iterative nucleic acid-guided nuclease editing, comprising: (i) providing a plurality of edited cells, the edited cells modified during an initial round of editing to include a first genomic edit, the edited cells comprising: a first editing vector comprising a first editing cassette and a first selectable marker; (ii) transforming the cells with a second editing vector, the second editing vector comprising: a second editing cassette, a second selectable marker, and a first nucleic acid sequence coding for a first curing gRNA configured to target the first selectable marker of the first editing vector; (iii) providing conditions for curing the first editing vector in the plurality of edited cells with a curing efficiency of at least 99%, wherein curing the first editing vector comprises cleaving the first editing vector at the first selectable marker with a nucleic acid-guided nuclease guided by the first curing gRNA; (iv) providing conditions for editing the plurality of edited cells with the second editing cassette, wherein the second editing cassette is configured to further modify the edited cells to include a second genomic edit; (v) transforming the cells with a third editing vector, the third editing vector comprising: a third editing cassette, a third selectable marker, and a second nucleic acid sequence coding for a second curing gRNA configured to target the second selectable marker of the second editing vector; (vi) providing conditions for curing the second editing vector in the plurality of edited cells with a curing efficiency of at least 99%, wherein curing the second editing vector comprises cleaving the second editing vector at the second selectable marker with the nucleic acid-guided nuclease guided by the second curing gRNA; and (vii) providing conditions for editing the plurality of edited cells with the third editing cassette, wherein the third editing cassette is configured to further modify the edited cells to include a third genomic edit.

In one aspect, the disclosure provides, and includes, a method for iterative nucleic acid-guided nuclease editing, comprising: (i) providing a plurality of edited cells, the edited cells modified during an initial round of editing to include a plurality of first genomic edits, the edited cells comprising: a plurality of first editing vectors, each vector of the plurality of first editing vectors comprising: at least one editing cassette of a library of first editing cassettes, wherein one or more editing cassettes of the library of first editing cassettes comprises a unique edit of the plurality of first genomic edits; and a first selectable marker; (ii) transforming the cells with a plurality of second editing vectors, each vector of the second editing vectors comprising: at least one editing cassette of a library of second editing cassettes, wherein one or more editing cassettes of the library of second editing cassettes comprises a unique edit of a plurality of second genomic edits; a second selectable marker; and a first nucleic acid sequence coding for a first curing gRNA configured to target the first selectable marker of the plurality of first editing vectors; (iii) providing conditions for curing the plurality of first editing vectors in the plurality of edited cells, wherein curing the plurality of first editing vectors comprises cleaving the plurality of first editing vectors at the first selectable marker with a nucleic acid-guided nuclease guided by the first curing gRNA of the plurality of second editing vectors; and (iv) providing conditions for editing the plurality of edited cells with the plurality of second editing vectors, wherein the plurality of second editing vectors are configured to further modify the edited cells to include the plurality of second genomic edits.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIGS. 1A and 1B respectively depict graphs of a model numerical simulation demonstrating the effects of curing efficiency and transformation efficiency on editing efficiency in an iterative editing process.

FIG. 2 is a simplified block diagram of an example of a method for iterative editing and curing of live cells, according to certain aspects of the present disclosure.

FIGS. 3A and 3B are simplified diagrams of examples of iterative methods for editing and curing, according to certain aspects of the present disclosure.

FIGS. 4A and 4B schematically depict examples of plasmid architectures of editing and engine vectors for use with the methods of FIG. 2 and FIGS. 3A-3B, according to certain aspects of the present disclosure.

FIG. 5 schematically illustrates an iterative curing system architecture comprising a plurality of editing vectors, wherein each editing vector is configured to cure another editing vector in the system, according to certain aspects of the present disclosure.

FIG. 6 depicts several graphs demonstrating the self-targeting activity of various curing gRNAs. Each point on the graph represents a different gRNA sequence. The best gRNAs for curing exhibit the largest depletion (lowest depletion scores, shown on log₂ scale) on this graph.

FIG. 7 is a graph demonstrating curing efficiency of individual gRNAs selected from the curing gRNAs in FIG. 6 for high self-targeting activity.

FIG. 8 is a graph demonstrating the difference in curing efficiency of systems utilizing targeting gRNAs designed to target a previously-transformed plasmid versus systems utilizing non-specific, non-targeting gRNAs.

FIG. 9 is a graph demonstrating the effect of DNA concentration on curing efficiency.

FIG. 10 is a graph demonstrating the curing efficiency of an iterative curing system during iterative rounds of curing on pooled samples.

FIG. 11 is a graph demonstrating curing technology used to rapidly increase the production of a proprietary molecule in E. coli by over 750 fold in seven rounds of iterative editing.

FIG. 12 is a graph demonstrating the fold improvement (FI) of samples edited in round six of iterative editing compared to the positive control (top producer from the previous round five of iterative editing).

It should be understood that the drawings are not necessarily to scale, and that like reference numbers refer to like features.

### DETAILED DESCRIPTION

All the functionalities described in connection with one aspect are intended to be applicable to the additional aspects described herein except where expressly stated or where the feature or function is incompatible with the additional aspects. For example, where a given feature or function is expressly described in connection with one aspect but not expressly mentioned in connection with an alternative aspect, it should be understood that the feature or function may be deployed, utilized, or implemented in connection with the alternative aspect unless the feature or function is incompatible with the alternative aspect.

The practice of the techniques described herein may employ, unless otherwise indicated, techniques and descriptions of organic chemistry, polymer technology, molecular biology (including recombinant techniques), cell biology, biochemistry and sequencing technology, which may be within the skill of those who practice in the art. Although certain of these techniques are known, combinations of these techniques may not be known. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; Berg et al. (2002) Biochemistry, 5^{th} Ed., W.H. Freeman Pub., New York, N.Y.; all of which are herein incorporated in their entirety by reference for all purposes. CRISPR-specific techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural references unless the context clearly dictates otherwise. Thus, for example, reference to “an oligonucleotide” refers to one or more oligonucleotides, and reference to “an automated system” includes reference to equivalent steps and methods for use with the system known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit aspects of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit aspects of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference herein in their entireties.

When a range of numbers is provided herein the range is understood to be inclusive of the edges of the range as well as any number between the defined edges of the range. For example, “between 1 and 10” includes any number between 1 and 10, as well as the number 1 and number 10.

The term “about” means plus or minus 10% of the numerical value of the number with which it is being used. For example, “about 100” refers to numbers between (and including) 90 and 110.

When a grouping of alternatives is presented, any and all combinations of the members that make up that grouping of alternatives is specifically envisioned. For example, if an item is selected from a group consisting of A, B, C, and D, the inventors specifically envisions each alternative individually (e.g. A alone, B alone, etc.), as well as combinations such as A, B, and D; A and C; B and C; etc.

The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

In the following description, numerous specific details are set forth to provide a more thorough understanding of the present invention. However, it will be apparent to one of ordinary skill in the art that the present invention may be practiced without one or more of these specific details. In other instances, well-known features and procedures well known to those skilled in the art have not been described in order to avoid obscuring the invention.

The term “amino acid” refers to naturally occurring and synthetic amino acids, as well as amino acid analogs that function similarly to naturally occurring amino acids.

The terms “cassette,” “expression cassette,” “editing cassette,” “CREATE cassette,” “CREATE editing cassette,” “CREATE fusion editing cassette,” or “CF editing cassette” in the context of the current methods and compositions refer to a nucleic acid molecule comprising a coding sequence for transcription of a guide nucleic acid (gRNA) to facilitate editing of one or both DNA strands in a nucleic acid-guided nuclease system. In certain aspects, “CF editing cassette” refers to a nucleic acid molecule comprising a coding sequence for transcription of a guide nucleic acid or gRNA covalently linked to a coding sequence for transcription of a repair template.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. The terms “percent complementarity” or “percent complementary” as used herein in reference to two nucleotide sequences is similar to the concept of percent identity but refers to the percentage of nucleotides of a query sequence that optimally base-pair or hybridize to nucleotides in a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (e.g., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%. In general, a nucleic acid includes a nucleotide sequence described as having a “percent complementarity” or being a “percent complementary” to a specified second nucleotide sequence. For example, a nucleotide sequence may have 70%, 80%, 90%, 95%, 99%, or 100% complementarity to a specified second nucleotide sequence, indicating that, for example, 7 of 10, 8 of 10, 9 of 10, 19 of 20, 99 of 100, or 10 of 10 nucleotides, respectively, of a sequence are complementary to the specified second nucleotide sequence. For example, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed, and—for some components—translated in an appropriate host cell.

The terms “CREATE fusion gRNA” or “CFgRNA” refer to a gRNA engineered to function with a nucleic acid-guided nickase/reverse transcriptase fusion enzyme (a “nickase-RT fusion”) where the CFgRNA is designed to bind to and facilitate editing of one or both DNA strands in a target locus of a cell genome. In certain aspects, “CREATE fusion gRNA” or “CFgRNA” refer to one of two gRNAs engineered to function with a nucleic acid-guided nickase/reverse transcriptase fusion enzyme (a “nickase-RT fusion”) where the two CFgRNAs are designed to bind to and edit opposite DNA strands in a target locus. The two CFgRNAs specific to a target locus have regions of complementarity to one another at least at the site of the edit and preferably at regions 5′ and 3′ to the site of the edit. The term “complementary CFgRNAs” refers to two CFgRNAs engineered to bind to opposite DNA strands in a target locus which often create the complementary edit at a site in the target locus.

The terms “CREATE fusion editing system” or “CF editing system” refer to the combination of a nucleic acid-guided nickase enzyme/reverse transcriptase fusion protein (“CREATE fusion enzyme” or “CF enzyme” or “nickase-RT fusion enzyme”) and a CREATE fusion editing cassette (“CF editing cassette”) to effect editing in live cells.

As used herein, “enrichment” refers to enriching for edited cells by singulation, inducing editing, and growth of singulated cells into terminal-sized colonies (e.g., saturation or normalization of colony growth).

The term “gene” refers to a segment of DNA involved in producing a polypeptide chain. It may include regions preceding and following a coding region (leader and trailer, respectively), as well as intervening sequences (introns) between individual coding segments (exons).

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease. The term “editing gRNA” refers to the gRNA used to edit a target sequence in a cell, typically a sequence endogenous to the cell. The term “curing gRNA” refers to the gRNA used to target a curing target sequence on an editing or engine vector.

The term “heterologous” refers to the relationship between two or more nucleic acids or protein sequences from different sources, or the relationship between a protein (or nucleic acid) and a host cell from different sources. For example, if the combination of a nucleic acid and a host cell is usually not naturally occurring, the nucleic acid is heterologous to the host cell. A particular sequence is “heterologous” to the cell or organism into which it is inserted.

“Homology” or “identity” or “similarity” refers to sequence similarity between two peptides or, more often in the context of the present disclosure, between two nucleic acid molecules. The term “homologous region” or “homology arm” refers to a region on a donor DNA or repair template with a certain degree of homology with a target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

The terms “percent identity” or “percent identical” as used herein in reference to two or more nucleotide or amino acid sequences is calculated by (i) comparing two optimally aligned sequences (nucleotide or amino acid) over a window of comparison (the “alignable” region or regions), (ii) determining the number of positions at which the identical nucleic acid base (for nucleotide sequences) or amino acid residue (for proteins and polypeptides) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to amino acids it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

For optimal alignment of sequences to calculate their percent identity, various pair-wise or multiple sequence alignment algorithms and programs are known in the art, such as ClustalW or Basic Local Alignment Search Tool® (BLAST™), etc., that can be used to compare the sequence identity or similarity between two or more nucleotide or amino acid sequences. Although other alignment and comparison methods are known in the art, the alignment and percent identity between two sequences (including the percent identity ranges described above) can be as determined by the ClustalW algorithm, see, e.g., Chenna et al., “Multiple sequence alignment with the Clustal series of programs,” Nucleic Acids Research 31: 3497-3500 (2003); Thompson et al., “Clustal W: Improving the sensitivity of progressive multiple sequence alignment through sequence weighting, position-specific gap penalties and weight matrix choice,” Nucleic Acids Research 22: 4673-4680 (1994); Larkin M A et al., “Clustal W and Clustal X version 2.0,” Bioinformatics 23: 2947-48 (2007); and Altschul et al. “Basic local alignment search tool.” J. Mol. Biol. 215:403-410 (1990), the entire contents and disclosures of which are incorporated herein by reference.

The term “nucleic acid” or “polynucleotide” refers to deoxyribonucleic acids (DNA) or ribonucleic acids (RNA) and polymers thereof in either single- or double-stranded form. Unless otherwise indicated, the terms encompass nucleic acids containing known analogues or natural nucleotides that have similar binding properties as the reference nucleic acid and are metabolized in a manner similar to naturally occurring nucleotides. Unless otherwise indicated, in addition to the sequence specifically stated, a particular nucleic acid sequence also implicitly encompasses conservatively modified variants thereof (e.g., degenerate codon substitutions), alleles, orthologues, SNPs, and complementary sequences. The term nucleic acid is used interchangeably with DNA, RNA, cDNA, gene, and mRNA encoded by a gene.

As used herein, “nucleic acid-guided nickase/reverse transcriptase fusion” or “nickase-RT fusion” refers to a nucleic acid-guided nickase—or nucleic acid-guided nuclease or CRISPR nuclease that has been engineered to act as a nickase rather than a nuclease that initiates double-stranded DNA breaks—where the nucleic acid-guided nickase is fused to a reverse transcriptase, which is an enzyme used to generate cDNA from an RNA template. In certain aspects, “nucleic acid-guided nickase/reverse transcriptase fusion” or “nickase-RT fusion” refers to two or more nucleic acid-guided nickases—or nucleic acid-guided nucleases or CRISPR nucleases that have been engineered to act as nickases rather than nucleases that initiate double-stranded DNA breaks—where the nucleic acid-guided nickases are fused to a reverse transcriptase. For information regarding nickase-RT fusions see, e.g., U.S. Pat. No. 10,689,669 and U.S. Ser. No. 16/740,421.

The term “nucleic acid-guided editing components” refers to one, some, or all of a nucleic acid-guided nuclease or nickase enzyme, a guide nucleic acid, and a repair template and/or donor nucleic acid.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “PAM mutation” refers to one or more edits to a target sequence that removes, mutates, or otherwise renders inactive a PAM or spacer region in the target sequence.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA. Promoters may be constitutive or inducible. In some aspects, a promoter is an endogenous promoter, synthetically produced, varied, or derived from a known or naturally occurring promoter sequence or other promoter sequence. In some aspects, a promoter is a constitutive promoter. In some aspects, a promoter is an inducible promoter. In some aspects, a promoter is a heterologous promoter. As used herein, a “constitutive promoter” refers to a promoter that is active in vivo at all times. Typically, the activity of a constitutive promoter is limited only by the presence of a suitable RNA polymerase at a suitable concentration.

As used herein, an “inducible promoter” refers to a regulated promoter that becomes active (e.g., it drives the expression of an operably linked sequence) in a cell in response to a specific stimulus.

As used herein, the terms “protein,” “peptide,” and “polypeptide” are used interchangeably herein and refer to a polymer of amino acid residues. In some aspects, proteins may or may not be made up entirely of amino acids transcribed by any class of any RNA polymerase I, II or III.

As used herein, the terms “repair template,” “homology arm,” or “donor nucleic acid” refer to 1) nucleic acid that is designed to facilitate introduction of a DNA sequence modification (insertion, deletion, substitution) into a locus by homologous recombination using nucleic acid-guided nucleases, or 2) a nucleic acid that serves as a template (including a desired edit) to be incorporated into target DNA by reverse transcriptase in a CREATE fusion editing (CFE) system. For homology-directed repair, a repair template or homology arm may have sufficient homology to the regions flanking the “cut site” or the site to be edited in the genomic target sequence. For template-directed repair, the repair template or homology arm has homology to the genomic target sequence except at the position of the desired edit although synonymous edits may be present in the homologous (e.g., non-edit) regions. The length of the repair template(s) or homology arm(s) will depend on, e.g., the type and size of the modification being made. In many instances and preferably, the repair template will have two regions of sequence homology (e.g., two homology arms) complementary to the genomic target locus flanking the locus of the desired edit in the genomic target locus. Typically, an “edit region” or “edit locus” or “DNA sequence modification” region—the nucleic acid modification that one desires to be introduced into a genome target locus in a cell (e.g., the desired edit)—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the target sequence.

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 may be employed. In other aspects, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+ cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2α; detectable by Mab-FITC); Methylguanine-DNA methyltransferase (MGMT; selectable by carmustine); rhamnose; and Cytidine deaminase (CD; selectable by Ara-C). “Selective medium” as used herein refers to cell growth medium to which has been added a chemical compound or biological moiety that selects for or against selectable markers.

The term “specifically binds” as used herein includes an interaction between two molecules, e.g., an engineered peptide antigen and a binding target, with a binding affinity represented by a dissociation constant of about 10⁻⁷ M, about 10⁻⁸ M, about 10⁻⁹ M, about 10⁻¹⁰ M, about 10⁻¹¹ M, about 10⁻¹² M, about 10⁻¹³ M, about 10⁻¹⁴ M, or about 10⁻¹⁵ M.

The terms “target genomic DNA locus”, “target editing locus”, “target locus”, “target sequence,” “cellular target locus,” or “genomic target locus” refer to any locus in vitro or in vivo, or in a nucleic acid (e.g., genome or episome) of a cell or population of cells, in which a change of at least one nucleotide is desired using a nucleic acid-guided nuclease editing system. The target sequence can be a genomic locus or extrachromosomal locus. The term “curing target sequence” refers to a sequence in an editing vector or engine vector that is cleaved or cut to cure or clear the vector. The term “target sequence” refers to either or both of a cellular target sequence and a curing target sequence.

The terms “transformation”, “transfection” and “transduction” refer to one or more processes of introducing exogenous DNA into cells.

The term “variant” may refer to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, BACs, YACs, PACs, synthetic chromosomes, and the like. In the present disclosure, a single vector may include a coding sequence for a nuclease or nickase and an editing cassette and/or a curing gRNA to be transcribed. In other aspects, however, two vectors—e.g., an engine vector comprising the coding sequence for the nuclease or nickase enzyme, and an editing vector, comprising the editing gRNA sequence and curing gRNA sequence to be transcribed—may be used.

The present disclosure provides systems, methods, and compositions for performing many rounds (e.g., iterations) of genomic editing utilizing large libraries of desired edits in each round. In certain examples, the present disclosure provides a system comprising a plurality of editing vectors that can be used in a defined and iterative process to enable several rounds of editing to be performed reliably and in rapid succession using genome-scale libraries of edits. As a result, the system enables rapid diversification and engineering of cell genomes in a manner that resembles traditional combinatorial protein engineering methodologies.

In certain examples, each editing vector of the system is designed to have “curing” activity encoded by a constitutively expressed gRNA that assembles with a nucleic acid-guided nuclease to actively target a selectable marker of one other vector in the set. Curing is a way to eliminate a prior editing vector-including the attendant gRNA and donor nucleic acids contained on the editing vector, as well as selection genes and other sequences contained on the editing vector, to permit a new editing vector to propagate within a cell without competition from the prior editing vector. Thus, when transformed into cells during a round of editing, each editing vector may eliminate, or “cure,” a corresponding editing vector from a previous round of editing. In certain examples, the editing vectors also share the same origin of replication, thereby facilitating mutually exclusive propagation for each newly transformed cell during iterative rounds of transformation and growth.

The systems, methods, and compositions described herein enable high curing efficiencies (e.g., >99%) during each round of an iterative editing process, thereby facilitating improved editing efficiencies (e.g., >70%) for each editing round for up to three, four, five, six, seven, eight, nine, or ten or more rounds of editing. Accordingly, the systems, methods, and compositions described herein satisfy or exceed the performance criteria necessary to truly unlock forward engineering at genome scale.

Thus, in certain aspects of the present disclosure, there is provided a method for iterative nucleic acid-guided nuclease editing, comprising: providing a plurality of edited cells, the edited cells modified during an initial round of editing to include a first genomic edit, the edited cells comprising: a first editing vector, the first editing vector comprising: a first editing cassette and a first selectable marker; transforming the cells with a second editing vector, the second editing vector comprising: a second editing cassette, a second selectable marker, wherein the second selectable marker is different from the first selectable marker, and a first nucleic acid sequence coding for a first curing gRNA configured to target the first selectable marker of the first editing vector; providing conditions for curing the first editing vector in the plurality of edited cells, wherein curing the first editing vector comprises cleaving the first editing vector at the first selectable marker with a nucleic acid-guided nuclease guided by the first curing gRNA; and, providing conditions for editing the plurality of edited cells with the second editing cassette, wherein the second editing cassette is configured to further modify the edited cells to include a second genomic edit.

In certain aspects of the present method, the method further comprises: transforming the cells with a third editing vector, the third editing vector comprising: a third editing cassette, a third selectable marker, wherein the third selectable marker is different from the second selectable marker, and a second curing gRNA configured to target the second selectable marker of the second editing vector; providing conditions for curing the second editing vector in the plurality of edited cells, wherein curing the second editing vector comprises cleaving the second editing vector at the second selectable marker with the nucleic acid-guided nuclease guided by the second curing gRNA; and, providing conditions for editing the plurality of edited cells with the third editing cassette, wherein the third editing cassette is configured to further modify the edited cells to include a third genomic edit.

In certain aspects of the present method, the method further comprises: transforming the cells with a fourth editing vector, the fourth editing vector comprising: a fourth editing cassette, a fourth selectable marker, wherein the fourth selectable marker is different from the third selectable marker, and a third curing gRNA configured to target the third selectable marker of the third editing vector; providing conditions for curing the third editing vector in the plurality of edited cells, wherein curing the third editing vector comprises cleaving the third editing vector at the third selectable marker with the nucleic acid-guided nuclease guided by the third curing gRNA; and, providing conditions for editing the plurality of edited cells with the fourth editing cassette, wherein the fourth editing cassette is configured to further modify the edited cells to include a fourth genomic edit.

In some aspects, the method facilitates a curing efficiency of prior editing vectors, e.g., the first editing vector, that is greater than 80%, greater than 85%, greater than 90%, greater than 95%, or greater than 99%. In some aspects, the method facilitates a curing efficiency of prior editing vectors of at least 80%, at least 85%, at least 90%, at least 95%, or at least 99%.

In some aspects, the method facilitates a curing efficiency of prior editing vectors, e.g., the first editing vector, that is greater than 99%, such as a curing efficiency of greater than 99.1%, 99.2%, 99.3%, 99.4%, 99.5%, 99.6%, 99.7%, 99.8%, 99.9%, 99.99%, 99.999%, or 99.9999%.

In some aspects, the method facilitates a curing efficiency of the second editing vector that is greater than 80%, greater than 85%, greater than 90%, greater than 95%, or greater than 99%. In some aspects, the method facilitates a curing efficiency of the second editing vector of at least 80%, at least 85%, at least 90%, at least 95%, or at least 99%.

In some aspects, the method facilitates a curing efficiency of the second editing vector that is greater than 99%, such as a curing efficiency of greater than 99.1%, 99.2%, 99.3%, 99.4%, 99.5%, 99.6%, 99.7%, 99.8%, 99.9%, 99.99%, 99.999%, or 99.9999%.

In some aspects, the method facilitates a curing efficiency of the third editing vector that is greater than 80%, greater than 85%, greater than 90%, greater than 95%, or greater than 99%. In some aspects, the method facilitates a curing efficiency of the third editing vector of at least 80%, at least 85%, at least 90%, at least 95%, or at least 99%.

In some aspects, the method facilitates a curing efficiency of the third editing vector that is greater than 99%, such as a curing efficiency of greater than 99.1%, 99.2%, 99.3%, 99.4%, 99.5%, 99.6%, 99.7%, 99.8%, 99.9%, 99.99%, 99.999%, or 99.9999%.

In some aspects, the first, second, third, fourth, fifth, sixth, seventh, eighth and so on editing vectors facilitate a curing efficiency of the prior editing vector that is greater than 80%, greater than 85%, greater than 90%, greater than 95%, or greater than 99%. In some aspects, the first, second, third, fourth, fifth, sixth, seventh, eighth and so on editing vectors facilitate a curing efficiency of the prior editing vector of at least 80%, at least 85%, at least 90%, at least 95%, or at least 99%.

In some aspects, the first, second, third, fourth, fifth, sixth, seventh, eighth and so on editing vectors facilitate a curing efficiency of the prior editing vectors that is greater than 99%, such as a curing efficiency of greater than 99.1%, 99.2%, 99.3%, 99.4%, 99.5%, 99.6%, 99.7%, 99.8%, 99.9%, 99.99%, 99.999%, or 99.9999%.

In some aspects, the first editing vector and the second editing vector comprise the same type of origin of replication (i.e., “replication origin”). In some aspects, the origin of replication is a temperature-sensitive origin of replication, e.g., a SC101 origin of replication.

In some aspects, the first, second, third or fourth selectable marker comprise antibiotic resistance genes. In specific aspects, the first selectable marker and/or the second selectable marker and/or the third selectable marker and/or the fourth selectable marker comprise one of an ampicillin/carbenicillin resistance gene, an ampicillin resistance gene, a carbenicillin resistance gene, a kanamycin resistance gene, a chloramphenicol resistance gene, an erythromycin resistance gene, a tetracycline resistance gene, a gentamicin resistance gene, a bleomycin resistance gene, a streptomycin resistance gene, a rifampicin resistance gene, a puromycin resistance gene, a hygromycin resistance gene, a blasticidin resistance gene, or other suitable antibiotic resistance gene.

In some aspects, the methods comprise transforming the cells with first, second, third, fourth, fifth, sixth, seventh, eighth and so on editing vectors, wherein the selectable markers used on the editing vector in adjacent rounds are different; that is, the selectable markers used on the first and second editing vectors are different; the selectable markers used on the second and third editing vectors are different; the selectable marker used on the third and fourth editing vectors are different, and so on. However, in certain aspects, the selectable markers used on the first and third editing vectors are the same; the selectable markers used on the second and fourth editing vectors are the same; the selectable markers used on the third and fifth editing vectors are the same; the selectable markers used on the fourth and sixth editing vectors are the same, and so on.

In some aspects, the first curing gRNA in the second editing vector is under the control of a constitutive promoter. In other aspects, the first curing gRNA in the second editing vector is under the control of an inducible promoter. In some aspects, the first curing gRNA comprises a region of complementarity to a sequence (e.g., a target curing sequence) of the first selectable marker in the first editing vector to facilitate cutting or curing of the target curing sequence and thus facilitate curing of the first editing vector.

In some aspects, providing conditions for curing an editing vector, e.g., the first editing vector in the plurality of edited cells comprises incubating the cells at a temperature between about 12-36° C. for a time period between 12-48 hours. In some aspects, providing conditions for curing a second, third, or fourth editing vector in the plurality of edited cells comprises incubating the cells at a temperature between about 12-36° C. for a time period between 12-48 hours. In some aspects, providing conditions for curing one or more editing vectors in the plurality of edited cells comprises incubating the cells at a temperature between about 12-36° C. for a time period between 12-48 hours.

In some aspects, the first editing cassette and/or the second editing cassette are under the control of an inducible promoter, such as an inducible pL promoter. In certain aspects, the first editing cassette and/or the second editing cassette are under the control of a constitutive promoter. In some aspects, the third editing cassette and/or the fourth editing cassette are under the control of an inducible promoter, such as an inducible pL promoter. In certain aspects, the third editing cassette and/or the fourth editing cassette are under the control of a constitutive promoter.

In some aspects, providing conditions for editing the plurality of cells with the second editing vector comprises induction of editing. In some aspects, editing may be induced via exposure of the cells to an elevated temperature, e.g., a temperature between about 36-48° C., to activate a promoter controlling transcription of the second editing cassette on the second editing vector.

In some aspects, an editing cassette comprises a nucleic acid coding for an editing gRNA covalently linked to a repair template for effecting an edit. In some aspects, the first editing cassette comprises a first nucleic acid sequence coding for a first editing gRNA covalently linked to a first repair template for effecting the first edit. In some aspects, the second editing cassette comprises a second nucleic acid sequence coding for a second editing gRNA covalently linked to a second repair template for effecting the second edit. Generally, the first and second editing gRNAs each comprise a region of complementarity to a sequence of a target editing locus in which an edit is to be incorporated.

In some aspects, an editing gRNA, e.g., the first or second editing gRNA, is a CREATE fusion gRNA (“CFgRNA,” defined infra), and an editing cassette, .e.g., the first or second editing cassette, is a CREATE fusion editing cassette (“CF editing cassette,” defined infra) comprising from 5′ to 3′: 1) a nucleic acid sequence coding for an editing gRNA having a region of complementarity to a sequence of a target editing locus in which an edit is to be incorporated, the editing gRNA comprising: a guide or spacer sequence, and a scaffold region recognized by a corresponding nuclease or nickase; and 2) a repair template covalently linked to the nucleic acid sequence coding for the editing gRNA and comprising an edit.

In some aspects, the repair template further comprises, in addition to a desired edit to be made to the target locus, an edit (e.g., 1, 2, 3, 4, 5, or up to 10 edits) to immunize a target editing locus to prevent re-nicking or re-cutting thereof. As discussed herein, in some aspects, an edit to immunize a target editing locus to prevent re-nicking is one that alters the proto-spacer adjacent motif (PAM) or spacer such that subsequent binding at the target editing locus by the nucleic acid-guided polypeptide (e.g., nuclease, nickase, inactive nuclease or inactive nickase) is impaired or prevented.

In some aspects, the editing cassette further comprises an RNA G-quadruplex region at a 3′ end of the repair template to stabilize the cassette and improve target nicking or cleavage efficiency without inducing off-target activity.

In some aspects, the editing cassette further comprises an amplification priming site or subpool primer binding sequence at a 3′ end thereof. In specific aspects, the editing cassette further comprises a melting temperature booster sequence at a 5′ end thereof, which is a short protective DNA buffer sequence. In addition, in specific aspects, the editing cassette comprises regions of homology to a vector for gap-repair insertion of the cassette into the vector, such as an editing vector or engine vector.

In some aspects, the editing cassette further comprises a barcode sequence.

In some aspects, a region of complementarity between the editing gRNA and a target editing locus is from 4-120 nucleotides in length, or from 5-80 nucleotides in length, or from 6-60 nucleotides in length, e.g., from 0-10 nucleotides in length, 10-20 nucleotides in length, 20-50 nucleotides in length, or 50-100 nucleotides in length.

In some aspects, the edit region of the repair template of the editing cassette is from 1-750 nucleotides in length, or from 1-500 nucleotides in length, or from 1-150 nucleotides in length, e.g., from 1-10 nucleotides in length, 10-20 nucleotides in length, 20-50 nucleotides in length, 50-100 nucleotides, 100-250 nucleotides, 250-500 nucleotides, or 500-750 or more nucleotides in length.

In some aspects, the edit region of the repair template of the editing cassette comprises two or more edits, or three or more edits, or four or more edits, or five or more edits.

In some aspects, the edit created by the editing cassette in a target editing locus includes one or more base swaps in the target locus. A “substitution” or “swap” refers to the replacement of one or more nucleotides or amino acids to a given polynucleotide or amino acid sequence, respectively, as compared to an endogenous reference polynucleotide or amino acid sequence. In some aspects, a “substitution allele” refers to a nucleic acid sequence at a particular locus comprising a substitution.

In some aspects, the edit created by the editing cassette in a target editing locus is an insertion in the target locus.

In some aspects, the edit created by the editing cassette is an insertion of recombinase sites, protein degron tags, promoters, terminators, alternative-splice sites, CpG islands, etc.

In some aspects, the edit created by the editing cassette in a target editing locus is a deletion in the target locus.

In some aspects, the editing cassette is designed to provide a deletion of from 1 to 750 nucleotides at a target locus. In some aspects, the editing cassette is designed to provide a deletion of from 1 to 10 nucleotides, from 10 to 20 nucleotides, from 20 to 50 nucleotides, from 50 to 100 nucleotides, from 100 to 200 nucleotides, from 200 to 500 nucleotides or from 250 to 750 nucleotides at a target editing locus.

In some aspects, the edit created is a deletion of introns, exons, repetitive elements, promoters, terminators, insulators, CpG islands, non-coding elements, retrotransposons, etc.

In some aspects, the edit comprises several types of edits and/or comprises more than one of one or more types of edits. For example, in some aspects, the edit comprises two or more base swaps (e.g., 2, 3, 4, 5, or from 1 to 20 base swaps), some or all of which can be adjacent to each other or nonadjacent to each other. In some aspects, the edit comprises one or more base swaps (e.g., 2, 3, 4, 5, or from 1 to 20 base swaps) and an insertion of one or more nucleotides (e.g., 2, 3, 4, 5, or from 1 to 20 nucleotides). In some aspects, the edit comprises one or more base swaps (e.g., 2, 3, 4, 5, or from 1 to 20 base swaps) and a deletion of one or more nucleotides (e.g., 2, 3, 4, 5, or from 1 to 20 nucleotides).

In some aspects, the edit created by the editing cassette in a target editing locus is in a coding region in the target editing locus.

In some aspects, the edit created by the editing cassette in a target editing locus is in a noncoding region in the target editing locus.

In certain aspects of the present method, the method further comprises transforming the edited cells with the nucleic acid-guided nuclease, or a sequence coding for the nucleic acid-guided nuclease.

In some aspects, the nuclease includes a MAD-series nuclease, nickase, or a variant (e.g., orthologue) thereof. In some aspects, the nuclease includes a MAD1, MAD2, MAD3, MAD4, MAD5, MAD6, MAD7, MAD8, MAD9, MAD10, MAD11, MAD12, MAD13, MAD14, MAD15, MAD16, MAD17, MAD18, MAD19, MAD20, MAD2001, MAD2007, MAD2008, MAD2009, MAD2011, MAD2017, MAD2019, MAD297, MAD298, MAD299, or other MAD-series nuclease, nickase, variants thereof, and/or combinations thereof.

In some aspects, the nuclease includes a Cas9 nuclease (also known as Csn1 and Csx12), nickase, or a variant thereof.

In some aspects, the nuclease includes C2c1, C2c2, C2c3, Cas1, Cas1B, Cas2, Cas3, Cas4, Cas5, Cas6, Cas7, Cas8, Cas10, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, or similar nuclease, nickase, variants thereof, and/or combinations thereof.

In some aspects, such as aspects wherein a CF editing cassette is utilized, the nuclease is a fusion protein—i.e., a nucleic acid-guided nickase/reverse transcriptase fusion enzyme (a “nickase-RT fusion”)—that retains certain characteristics of nucleic acid-directed nucleases (e.g., the binding specificity and ability to cleave one or more DNA strands in a targeted manner) combined with another enzymatic activity, namely, reverse transcriptase activity. The reverse transcriptase portion of the nickase-RT fusion may use a repair template of an editing cassette to synthesize and edit at a “flap” created by the nickase portion on one or both DNA strands of a target editing locus, thereby circumventing endogenous mismatch repair systems to incorporate an edit.

In some aspects, the nuclease is introduced into the plurality of edited cells on a vector and under the control of an inducible or constitutive promoter. In specific aspects, the nuclease is introduced into the edited cells on a vector further comprising an editing cassette and/or a curing gRNA (e.g., an editing vector). In specific aspects, the nuclease is introduced into the cells on an engine vector. The engine vector may be co-delivered or sequentially delivered with one or more of the editing vectors. In such aspects, the engine vector may further comprise a selectable marker and/or a lambda (k) Red recombineering system under the control of a promoter. The λ Red recombineering system works as a “band aid” or repair system for double-strand breaks in bacteria, and in some species of bacteria, the λ Red recombineering system (or some other recombineering system) must be present for the double-strand breaks that occur during editing to resolve. In, for example, yeast and other eukaryotic cells, however, recombineering systems are not required. In some aspects, the λ Red recombineering system is under the control of an inducible promoter, which may be a different inducible promoter than that driving transcription of, e.g., the nuclease and/or editing cassette(s), thus enabling the induction of the λ Red recombineering system separate from the nuclease and/or editing cassette(s).

In some aspects, the nuclease may introduced into the plurality of edited cells separately in protein form or as part of a complex.

In some aspects, the editing vectors and/or engine vectors are circular plasmids.

In some aspects, there is provided a library of vector or plasmid backbones, and/or a library of editing cassettes to be transformed into cells during one or more editing rounds. In some aspects, the utilization of editing cassettes and/or vector or plasmid backbones enables combinatorial or multiplex editing in the cells. A library of cassettes or vectors may comprise cassettes or vectors that have any combination of common elements and non-common or different elements as compared to other cassettes or vectors within the pool. For example, a library of editing cassettes can comprise common priming sites or common nick-to-edit or post-edit homology regions, while also containing non-common or unique edits. Combinations of common and non-common elements are advantageous for multiplexing or combinatorial techniques disclosed herein.

In some aspects, a library of cassettes comprises at least 2 cassettes, at least 3 cassettes, at least 4 cassettes, at least 5 cassettes, at least 10 cassettes, at least 100 cassettes, at least 1,000 cassettes, at least 10,000 cassettes, at least 100,000 cassettes, or at least 1,000,000 cassettes. In some aspects, a library of cassettes comprises from five to a 1,000,000 cassettes, from 100 to 500,000 cassettes, from 1,000 to 100,000 cassettes, from 1,000 to 10,000 cassettes, or from 10,000 to 50,000 cassettes.

In some aspects, one or more editing cassettes in a library of editing cassettes each comprise a different editing gRNA targeting a different target locus within the cell genome. In some aspects, one or more editing cassettes in a library of editing cassettes each comprise a different edit to be incorporated within the cell genome. In some aspects, one or more nucleic acids in a library of donor nucleic acids each comprise a different barcode to be incorporated within the cell genome.

In some aspects, there is provided a trackable library comprising a plurality of cassettes or vectors as disclosed herein. In some aspects, within the trackable library are distinct editing cassette and/or barcode combinations, which when sequenced upon editing, facilitate tracking of editing events in a population of cells. Accordingly, when edits and barcodes are incorporated into a target genome, the incorporation of an edit and barcode may be tracked based on a sequenced barcode.

In some aspects, there is provided a gene-wide or genome-wide library of cassettes or vectors comprising a cassettes as disclosed herein.

The methods of editing and curing of the present disclosure may be applied to many cell types including, e.g., prokaryotic, archaeal, and eukaryotic cells. In some aspects, the cells include mammalian cells. In some aspects, the cells include bacterial or fungal cells. In some aspects, the cells include yeast cells. In some aspects, the cells include E. coli cells.

### Nucleic Acid-Guided Genome Editing, Generally

The compositions and methods described herein are employed to allow one to perform nucleic acid nuclease-directed genome editing (e.g., CRISPR editing) to introduce desired edits to a population of live cells. Specifically, the compositions and methods presented herein facilitate editing nucleotide sequences in a population of cells in a targeted, multiplex, and iterative manner, where editing vectors from previous rounds of editing are cured (e.g., cleared) utilizing subsequent editing vectors introduced into the population of cells for later rounds of editing. An advantage of the present methods and compositions is that they allow one to perform iterative (i.e., recursive) rounds of CRISPR-type nucleic acid-guided nuclease genome-wide targeted editing with reduced risk of interference or competition for newly-introduced editing vectors from prior editing vectors.

In CRISPR editing generally, a nucleic acid-guided nuclease or CREATE fusion enzyme complexed with an appropriate synthetic guide nucleic acid in a cell can cut the genome of the cell at a desired location. The guide nucleic acid helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects and preferably, the guide nucleic acid is a single guide nucleic acid construct that includes both (1) a guide sequence capable of hybridizing to a genomic target locus, and (2) a scaffold sequence capable of interacting or complexing with a nucleic acid-guided nuclease.

In general, a guide nucleic acid (e.g., gRNA) complexes with a compatible nucleic acid-guided nuclease or CREATE fusion enzyme and can then hybridize with a target sequence, thereby directing the nuclease to the target sequence. A guide nucleic acid can be DNA or RNA; alternatively, a guide nucleic acid may comprise both DNA and RNA. In some aspects, a guide nucleic acid may comprise modified or non-naturally occurring nucleotides. In some aspects, a guide nucleic acid comprises RNA, and the gRNA may be encoded by a DNA sequence on a polynucleotide molecule such as a plasmid, linear construct, or the coding sequence may and preferably does reside within an editing cassette. Methods and compositions for designing and synthesizing editing cassettes and libraries of editing cassettes are described in U.S. Pat. No. 10,240,167; 10,266,849; 9,982,278; 10,351,877; 10,364,442; 10,435,715; 10,465,207; 10,669,559; 10,711,284; 10,731,180; and 11,078,498.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 55%, 60%, 65%, 70%, 75%, 80%, 85%, 90%, 92.5%, 95%, 97.5%, 99%, or more. Optimal alignment may be determined with the use of any suitable algorithm for aligning sequences (e.g., without being limiting, BLAST™). In some aspects, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some aspects, a guide sequence is less than about 75, 50, 45, 40, 35, 30, 25, 20 nucleotides in length. Preferably, the guide sequence is between 10 nucleotides and 30 nucleotides long, between 15 nucleotides and 20 nucleotides long, or 15, 16, 17, 18, 19, or 20 nucleotides in length.

In certain aspects of the present methods and compositions, the guide nucleic acids are provided as a sequence to be expressed from a plasmid or vector and comprises both the guide sequence and the scaffold sequence as a single transcript under the control of a promoter, e.g., an inducible or constitutive promoter. In certain aspects, the guide nucleic acid may be part of an editing cassette that encodes a repair template for effecting an edit in the cellular target sequence, and/or one or more homology arms. Alternatively, the guide nucleic acid may not be part of the editing cassette and instead may be encoded on the vector backbone, such as an editing plasmid backbone. For example, a sequence coding for a guide nucleic acid can be assembled or inserted into an editing vector backbone first, followed by insertion of the repair template in, e.g., an editing cassette. In other cases, the repair template in, e.g., an editing cassette can be inserted or assembled into an editing vector backbone first, followed by insertion of the sequence coding for the guide nucleic acid. In certain aspects, the sequence encoding the guide nucleic acid and repair template are located together in a rationally designed editing cassette and are simultaneously inserted or assembled via gap repair into a linear vector or backbone to create an editing vector.

The guide nucleic acids are engineered to target a desired target sequence (either a cellular “editing” target sequence or a curing target sequence on a vector) by altering the guide sequence so that the guide sequence is complementary to a desired target sequence, thereby allowing hybridization between the guide sequence and the target sequence. The target sequence can be any polynucleotide endogenous or exogenous to a prokaryotic or eukaryotic cell, or in vitro. For example, the target sequence can be a polynucleotide residing in the nucleus of a eukaryotic cell. A target sequence can be a sequence encoding a gene product (e.g., a protein, a non-coding sequence (e.g., a regulatory polynucleotide, an intron, a proto-spacer adjacent motif (PAM) sequence, or “junk” DNA), or a curing target sequence in an editing vector. In the present description, the target sequence for one of the gRNAs, the curing gRNA, is on the editing vector.

In general, to generate an edit in the target sequence, a gRNA/nuclease complex binds to the target sequence as determined by the guide RNA, and the nuclease or CF enzyme recognizes a PAM sequence adjacent to or in proximity to the target sequence. The precise preferred PAM sequence and length requirements for different nucleic acid-guided nucleases vary; however, PAMs typically are 2-10 or so base-pairs in length and, depending on the nuclease, can be 5′ or 3′ to the target sequence. Engineering of the PAM-interacting domain of a nucleic acid-guided nuclease may allow for alteration of PAM specificity, improve target site recognition fidelity, decrease target site recognition fidelity, or increase the versatility of a nucleic acid-guided nuclease.

In most aspects, genome editing of a cellular target sequence both introduces a desired DNA change to a cellular target sequence (an “intended” edit), e.g., the genomic DNA of a cell, and removes, mutates, or renders inactive a PAM region in the cellular target sequence (an “immunizing edit”), thereby rendering the target site immune to further nuclease binding. Rendering the PAM at the cellular target sequence inactive precludes additional editing of the cell genome at that cellular target sequence, e.g., upon subsequent exposure to a nucleic acid-guided nuclease complexed with a synthetic guide nucleic acid in later rounds of editing. Thus, cells having the desired cellular target sequence edit and an altered PAM can be selected for by using a nucleic acid-guided nuclease complexed with a synthetic guide nucleic acid complementary to the cellular target sequence. Cells that did not undergo the first editing event may be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

As for the nuclease or CF enzyme component of the nucleic acid-guided nuclease or CF enzyme, a polynucleotide sequence encoding the nucleic acid-guided nuclease or CF enzyme can be codon optimized for expression in particular cell types, such as bacterial, yeast, and, here, mammalian cells. The choice of the nucleic acid-guided nuclease or CF enzyme to be employed depends on many factors, such as what type of edit is to be made in the target sequence and whether an appropriate PAM is located close to the desired target sequence. Nucleases of use in the methods described herein include but are not limited to Cas9, Cas12/Cpf1, MAD2, MAD7@, MAD2007 or other MADZYME® and MADZYME® systems (see U.S. Pat. No. 10,604,746; 10,655,114; 10,649,754; 10,876,102; 10,833,077; 11,053,485; 10,704,022; 10,745,678; 10,724,021; 10,767,169; 10,870,761; 10,011,849; 10,435,714; 10,626,416; 9,982,279; and 10,337,028; and U.S. Ser. Nos. 16/953,253; 17/374,628; 17,200,074; 17,200,089; 17/200,110; 16/953,233; 17/463,498; 63/134,938; 16/819,896; 17/179,193; and 16/421,783 for sequences and other details related to engineered and naturally-occurring MADzymes). CF enzymes typically comprise a CRISPR nucleic acid-guided nuclease engineered to cut one DNA strand in the target DNA rather than making a double-stranded cut, and the nickase portion is fused to a reverse transcriptase. In specific aspects, the one or more nickases include MAD7 nickase, MAD2001 nickase, MAD2007 nickase, MAD2008 nickase, MAD2009 nickase, MAD2011 nickase, MAD2017 nickase, MAD2019 nickase, MAD297 nickase, MAD298 nickase, MAD299 nickase, or other MAD-series nickases, variants thereof, and/or combinations thereof as described in U.S. Pat. Nos. 10,883,077; 11,053,485; 11,085,030; 11,200,089; 11,193,115; and U.S. Ser. No. 17/463,498. A coding sequence for a desired nuclease or CF enzyme may be on an “engine vector” along with other desired sequences such as a selective marker(s), or a coding sequence for the desired nuclease or nickase may reside on the editing vector or may be transfected into a cell as a protein.

Another component of the nucleic acid-guided nuclease system or CF system is the repair template comprising homology to the cellular target sequence. The repair template typically is designed to serve as a template for homologous recombination with a cellular target sequence cleaved by the nucleic acid-guided nuclease, or the repair template serves as the template for template-directed repair via the CF enzyme, as a part of the gRNA/nuclease complex. For the present methods and compositions, the repair template typically is on the same vector and, in certain aspects, in the same editing cassette, as a guide nucleic acid for editing, and may be under the control of the same promoter as the editing gRNA (that is, a single promoter driving the transcription of both the editing gRNA and the repair template). A repair template polynucleotide may be of any suitable length, such as about or more than about 20, 25, 50, 75, 100, or more nucleotides in length. In certain preferred aspects, the repair template can be provided as an oligonucleotide of between 20-100 nucleotides, such as between 30-75 nucleotides. When optimally aligned, the repair template overlaps with (is complementary to) the cellular target sequence by, e.g., about 20, 25, 30, 35, 40, 50, 60, 70, 80, 90 or more nucleotides.

The repair template generally comprises two regions that are complementary to a portion of the cellular target sequence (e.g., homology arms). In certain aspects of the present methods and compositions, the two homology arms flank an intended edit, e.g., at least one alteration as compared to the cellular target sequence, such as a DNA sequence insertion, which may be part of the repair template. In certain aspects, the repair template comprises two homology arms that do not flank the intended edit. In such aspects, the homology arms may be encoded on an editing vector backbone, or in an editing cassette with the edit.

Inducible editing is advantageous in that cells can be grown for several to many cell doublings before editing is initiated, which increases the likelihood that cells with edits will survive, as the double-strand cuts caused by active editing are largely toxic to the cells. This toxicity results both in cell death in the edited colonies, as well as possibly a lag in growth for the edited cells that do survive but must repair and recover following editing. However, once the edited cells have a chance to recover, the size of the colonies of the edited cells will eventually catch up to the size of the colonies of unedited cells. It is this toxicity, however, that is exploited herein to perform curing.

An editing cassette may further comprise one or more primer binding sites. The primer binding sites are used to amplify the editing cassette by using oligonucleotide primers as described infra and may be biotinylated or otherwise labeled.

In addition, the editing cassette may comprise a barcode. A barcode is a unique DNA sequence that corresponds to the repair template such that the barcode can identify the edit made to the corresponding cellular target sequence. The barcode typically comprises four or more nucleotides. In some aspects, the editing cassettes comprise a collection or library of editing gRNAs and of repair templates representing, e.g., gene-wide or genome-wide libraries of editing gRNAs and donor nucleic acids. The library of editing cassettes is cloned into vector backbones where, e.g., each different repair template/donor nucleic acid is associated with a different barcode.

In certain aspects, the plasmid and/or vector encoding components of the nucleic acid-guided nuclease system, e.g., the editing vector, further encodes a nucleic acid-guided nuclease comprising one or more nuclear localization sequences (NLSs), such as about or more than about 1, 2, 3, 4, 5, 6, 7, 8, 9, 10, or more NLSs, particularly as an element of the nuclease sequence. In some aspects, the engineered nuclease comprises NLSs at or near the amino-terminus, NLSs at or near the carboxy-terminus, or a combination.

In certain aspects, the vectors further comprise one or more selectable markers to enable artificial selection of cells undergoing editing and/or curing events. For example, in certain aspects, the editing vectors encode for one or more antibiotic resistance genes, such as ampicillin/carbenicillin and chloramphenicol resistance genes, thereby facilitating enrichment for cells undergoing editing and/or curing events via depletion of the cell population. In other examples, vectors may include an integrated GFP gene to enable phenotypic detection of editing and/or curing events by flow cytometry, fluorescent cell imaging, etc. In certain aspects and as described below, the one or more selectable markers may be further utilized as curing targets for curing gRNAs transformed into the cells in later rounds of editing. For example, a curing gRNA transformed in a later round of editing may be designed to target a sequence of a selectable marker found on an earlier-transformed editing vector. Accordingly, in addition to facilitating selection of edited and/or cured cells, the selectable markers may facilitate targeted curing of the cells.

In certain aspects, engine and editing vectors may further comprise control sequences operably linked to the component sequences to be transcribed. As described above, promoters driving transcription of one or more components of the nucleic acid-guided nuclease editing system may be inducible. A number of gene regulation control systems have been developed for the controlled expression of genes in plant, microbe, and animal cells, including mammalian cells, such as the pL promoter (induced by heat inactivation of the cI857 repressor), the pPhIF promoter (induced by the addition of 2,4 diacetylphloroglucinol (DAPG)), the pBAD promoter (induced by the addition of arabinose to the cell growth medium), and the rhamnose inducible promoter (induced by the addition of rhamnose to the cell growth medium). Other systems include the tetracycline-controlled transcriptional activation system (Tet-On/Tet-Off, Clontech, Inc. (Palo Alto, Calif.); Bujard and Gossen, PNAS, 89(12):5547-5551 (1992)), the Lac Switch Inducible system (Wyborski et al., Environ Mol Mutagen, 28(4):447-58 (1996); DuCoeur et al., Strategies 5(3):70-72 (1992); U.S. Pat. No. 4,833,080), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)) as well as others. In certain aspects of the present methods used in the modules and instruments described herein, at least one of the nucleic acid-guided nuclease editing components (e.g., the nuclease and/or the gRNA) is under the control of a promoter that is activated by a rise in temperature, as such a promoter allows for the promoter to be activated by an increase in temperature, and de-activated by a decrease in temperature, thereby “turning off” the editing process. Thus, in the scenario of a promoter that is de-activated by a decrease in temperature, editing in the cell can be turned off without having to change media; to remove, e.g., an inducible biochemical in the medium that is used to induce editing.

### Curing for Iterative Nucleic-Acid Guided Editing

“Curing” is a process in which a vector—here, e.g., the editing vector used in a prior round of editing—is removed from the cells being edited. Curing can be accomplished by various mechanisms, including (1) diluting the editing vector in the cell population via cell growth—that is, the more growth cycles the cells go through in medium without an antibiotic that selects for the editing vector, the fewer daughter cells will retain the editing or engine vector(s); (2) cleaving the editing vector using a curing gRNA on the engine or editing vectors, thereby rendering the editing vector nonfunctional; and (3) utilizing a heat-sensitive origin of replication on the editing vector.

When performed during an iterative editing process, curing permits a newly introduced editing vector to propagate within a cell with reduced competition from prior editing vectors. If the cell is left uncured, or if curing is performed with relatively low efficiency, propagation of a newly introduced editing vector may be limited as a result of the competition from persisting prior editing vectors, causing decreased editing efficiency during the current and any subsequent editing round. Accordingly, performing curing with high efficiency is key to obtaining high editing efficiency in multiple rounds of iterative editing.

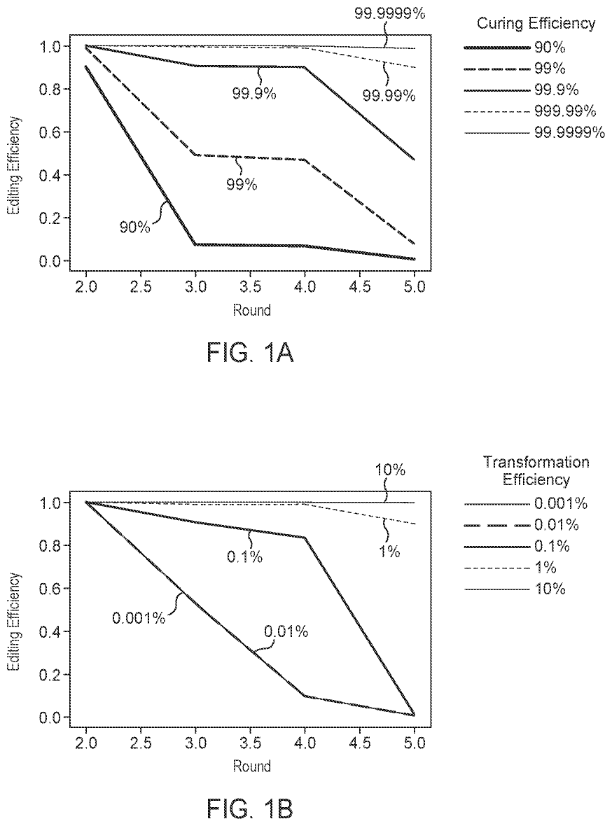

FIGS. 1A and 1B depict graphs respectively demonstrating the effects of curing efficiency and transformation efficiency on editing efficiency in an iterative editing process, as realized in a model numerical simulation prepared by the inventors of the present disclosure. In particular, FIGS. 1A and 1B simulate a five-round iterative editing process wherein a new editing vector is introduced, and a prior editing vector is cured, in each round of editing. The utilized model is based on a 2-antibiotic/4-selectable marker variant system as described elsewhere herein, although it can be generalizable for any configuration of antibiotics and antibiotic markers.

In FIG. 1A, curing efficiency for each sample was set at either 90%, 99%, 99.9%, 99.99%, or 99.9999% per round. Transformation efficiency of each editing vector was set to 1% of cells in each round, or 0.01 cells/round, and it was assumed that any uncured cells after each round were incompetent for the next round of editing with a newly transformed editing vector. As shown, iterative editing efficiency is strongly dependent upon curing efficiency. In the simulation of FIG. 1A, low curing efficiency (e.g., 90%, 99%) allowed uncured cells to rapidly overtake the cell population, leading to severely reduced editing efficiency in subsequent rounds of editing. In contrast, a curing efficiency of 99.9% or greater provided meaningful (e.g., substantial) editing efficiencies in samples beyond four or more rounds of editing.

In FIG. 1B, curing efficiency for each sample was set to 99.99% per round, and transformation efficiency was varied between 10%, 1%, 0.1%, 0.01%, or 0.001%. Similar to FIG. 1A, it was assumed that any uncured cells after each round were incompetent for the next round of editing with a newly transformed editing vector. As shown, iterative editing efficiency is also strongly dependent upon transformation (uptake) efficiency. In the simulation of FIG. 1B, low transformation efficiency (e.g., 0.001%, 0.01%) lead to a large proportion of the cell population comprising uncured cells, thereby severely reducing editing efficiency in subsequent rounds of editing. In contrast, a transformation efficiency of 0.1% or greater provided meaningful editing efficiencies in samples beyond four or more rounds of editing.

In light of the above, the inventors of the present disclosure have discovered that a curing efficiency of >99% (of prior editing vectors), such as a curing efficiency of >99.99%, in combination with a relatively high vector transformation efficiency (e.g., >0.1%), is needed to successfully effect multiple rounds of iterative editing with meaningful results, and have developed systems, methods, and compositions as described herein that enable such high curing efficiencies during each round of an iterative editing process. Accordingly, the systems, methods, and compositions described herein facilitate improved editing efficiencies (e.g., >70%) for each editing round for up to three, four, five, six, seven, eight, nine, or ten or more iterative rounds of editing.

Thus, in certain aspects described herein, the present disclosure is drawn to transcribing a curing gRNA to cut or cleave a curing target sequence in an editing vector introduced into the cells for an earlier round of editing during an iterative editing process. The curing gRNA may be—and in the present methods is—located on an editing vector transformed into the cells for a later round of editing, thereby enabling curing of the earlier editing vector before initiation of the later round of editing. In certain aspects, the curing target sequence includes a sequence located within a selectable marker of the earlier editing vector, thereby allowing the selectable marker to function not only as a means for selecting cells transformed with the editing vector, but also as a means for enabling targeted curing of the same. Accordingly, the present disclosure provides systems, methods, and compositions for performing high efficiency curing with reduced process complexity.

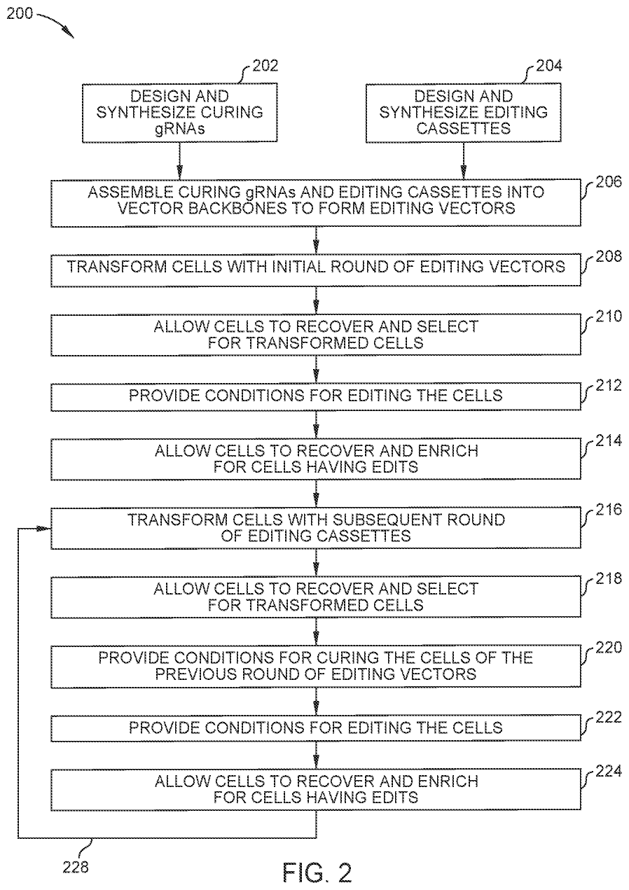

FIG. 2 illustrates a flow chart for exemplary method 200 for performing iterative nucleic acid-guided nuclease editing and curing according to aspects of the present disclosure. Method 200 facilitates multiple rounds of editing to effect multiple edits in cells with reduced risk of interference or competition for newly-introduced editing vectors from prior editing vectors. Thus, in certain examples, method 200 may be utilized to prepare diverse populations of cells with multiple edits in each cell, or to engineer a cell strain with a desired production phenotype. However, other applications are also contemplated.

Looking at FIG. 2, method 200 begins by designing and synthesizing nucleic acid-guided nuclease editing components for the iterative editing process. In particular, method 200 begins by designing and synthesizing curing gRNAs 202 and editing cassettes 204.

Curing gRNAs 202 are designed to target a desired target sequence (i.e., a curing target sequence) on one or more editing vectors to be utilized during the method 200. In certain aspects, the curing target sequence is a sequence of a selectable marker integrated onto the editing vector(s), such as an antibiotic-resistance gene. Thus, the curing gRNAs 202 may comprise a guide sequence that is complementary to at least a portion of the selectable marker sequence on the editing vector(s), thereby allowing hybridization between the curing gRNA 202 and the selectable marker on the editing vector(s).

Editing cassettes 204 may each be designed to comprise at least an editing gRNA sequence paired with a repair template sequence. In certain aspects, the editing gRNA and repair template may be covalently linked. In further aspects, the editing cassettes 204 include other desired sequences, such as a barcode, primer amplification sites, and the like. Methods and compositions particularly favored for designing and synthesizing editing cassettes are described in U.S. Pat. Nos. 10,240,167; 10,266,849; 9,982,278; 10,351,877; 10,364,442; 10,435,715; 10,465,207; 10,669,559; 10,711,284; 10,731,180; and 11,078,498.

Once the individual curing gRNAs and editing cassettes have been designed and synthesized, the curing gRNAs and individual cassettes are amplified (e.g., using PCR), purified, and assembled into vector backbones 206 to produce editing vectors (e.g., a library of editing vectors), each of which comprise an editing cassette or an editing cassette and a curing gRNA. A number of methods may be used to assemble the curing gRNAs and editing cassettes, including isothermal assembly, CPEC, SLIC, ligase cycling, etc. Additional assembly methods include gap repair in yeast (Bessa, Yeast, 29(10):419-23 (2012)), gateway cloning (Ohtsuka, Curr Pharm Biotechnol, 10(2):244-51 (2009); U.S. Pat. No. 5,888,732 to Hartley et al.; U.S. Pat. No. 6,277,608 to Hartley et al.; and topoisomerase-mediated cloning (Udo, PLoS One, 10(9):e0139349 (2015)); U.S. Pat. No. 6,916,632 B2 to Chestnut et al. These and other nucleic acid assembly techniques are described, e.g., in Sands and Brent, Curr Protoc Mol Biol., 113:3.26.1-3.26.20 (2016); Casini et al., Nat Rev Mol Cell Biol., (9):568-76 (2015); and Patron, Curr Opinion Plant Biol., 19:14-9 (2014)). Vector backbones chosen for the methods herein may vary depending on the type of cells being edited, where the vectors include, e.g., plasmids, BACs, YACs, viral vectors and synthetic chromosomes. The vector backbones may include one or more promoters for driving transcription of the curing gRNAs and/or editing cassettes, as well as one or more selectable markers, such as antibiotic resistance genes.

Once the editing vectors are assembled, an initial editing vector is transformed into cells 208. In certain aspects, the initial editing vector comprises both an editing cassette and a curing gRNA. In certain aspects, however, the initial editing vector comprises and editing cassette and no curing gRNA, as the cells have not been previously transformed with other editing vectors that require curing prior to the ensuing editing round.

As used herein, transformation is intended to generically include a variety of art-recognized techniques for introducing an exogenous nucleic acid sequence (e.g., an engine vector and/or editing vector) into a target cell, and the term “transformation” as used herein includes all transformation, transduction, and transfection techniques. Such methods include, but are not limited to, electroporation, lipofection, optoporation, injection, microprecipitation, microinjection, liposomes, particle bombardment, sonoporation, laser-induced poration, bead transfection, calcium phosphate or calcium chloride co-precipitation, or DEAE-dextran-mediated transfection. Cells can also be prepared for vector uptake using, e.g., a sucrose, sorbitol or glycerol wash. Additionally, hybrid techniques that exploit the capabilities of mechanical and chemical transfection methods can be used, e.g., magnetofection, a transfection methodology that combines chemical transfection with mechanical methods. In another example, cationic lipids may be deployed in combination with gene guns or electroporators. Suitable materials and methods for transforming or transfecting target cells can be found, e.g., in Green and Sambrook, Molecular Cloning: A Laboratory Manual, 4th ed., Cold Spring Harbor Laboratory Press, Cold Spring Harbor, N.Y. (2014).

Prior to transformation of the initial editing vector 208, cells of choice are grown in culture and, in certain aspects, are made electrocompetent. Cell culture is the process by which cells are grown under controlled conditions, almost always outside the cell's natural environment. For bacterial and yeast cells, the cells are typically grown in a defined medium in bulk culture. For mammalian cells, culture conditions typically vary somewhat for each cell type but generally include a medium and additives that supply essential nutrients such as amino acids, carbohydrates, vitamins, minerals, growth factors, hormones, and gases such as, e.g., O₂ and CO₂. In addition to providing nutrients, the medium typically regulates the physio-chemical environment via a pH buffer and most cells are grown at 37° C. Many mammalian cells require or prefer a surface or artificial substrate on which to grow (e.g., adherent cells), whereas other cells such as hematopoietic cells and some adherent cells can be grown in or adapted to grow in suspension. Adherent cells often are grown in 2D monolayer cultures in petri dishes or flasks, but some adherent cells can grow in suspension cultures to higher density than would be possible in 2D cultures. “Passages” generally refers to transferring a small number of cells to a fresh substrate with fresh medium, or, in the case of suspension cultures, transferring a small volume of the culture to a larger volume of medium.

The cells that can be edited according to the present methods include any prokaryotic, archaeal, or eukaryotic cells. For example, prokaryotic cells for use with the present illustrative aspects can be gram positive bacterial cells, e.g., Bacillus subtilis, or gram-negative bacterial cells, e.g., E. coli cells. Eukaryotic cells for use with the automated multi-module cell editing instruments of the illustrative aspects include any plant cells and any animal cells, e.g. fungal cells, insect cells, amphibian cells nematode cells, or mammalian cells.

In an aspect, a cell is selected from the group consisting of a bacterial cell, a fungal cell, an animal cell, and a plant cell. In an aspect, a bacterial cell is selected from the group consisting of an Escherichia sp. cell, a Bacillus sp. cell, a Streptomyces sp. cell, a Pseudomonas sp. cell, a Corynebacterium cell, and a Vibrio sp. cell. In an aspect, an Escherichia sp. cell is an Escherichia coli cell. In an aspect, a fungal cell is selected from the group consisting of Schizosaccharomyces pombe and Saccharomyces cerevisiae. In an aspect, an animal cell is selected from the group consisting of a mammal cell, a fish cell, a bird cell, a reptile cell, a worm cell, an insect cell, and an amphibian cell. In an aspect, a mammal cell is selected from the group consisting of a dog cell, a cat cell, a horse cell, a non-human primate cell, a rodent cell, and a lagomorph cell. In an aspect, a mammal cell is a human cell. In an aspect, a human cell is an ex vivo human cell.

As described elsewhere herein, the cells may be also transformed simultaneously with a separate engine vector expressing an editing nuclease; alternatively, the cells may already have been transformed with an engine vector configured to express the nuclease; that is, the cells may have already been transformed with an engine vector or the coding sequence for the nuclease may be stably integrated into the cellular genome such that only the editing vector needs to be transformed into the cells to facilitate editing after transformation 208.

Once transformed 208, the cells are allowed to recover and selection is optionally performed 210 to select for cells transformed with the initial editing vector, which comprise a selectable marker. Selectable markers and selection medium are employed to select for cells that have received the vector backbone. Commonly used selectable markers include drug selectable markers such as ampicillin/carbenicillin, kanamycin, chloramphenicol, erythromycin, tetracycline, gentamicin, bleomycin, streptomycin, puromycin, hygromycin, blasticidin, and GF18.

At a next step 212, conditions are provided to facilitate editing. “Providing conditions” includes incubation of the cells in appropriate medium and may also include providing conditions to facilitate, or even induce via an inducible promoter, transcription of the editing cassette and the nuclease, which may be on separate vectors. For example, if one or more components of the editing machinery (e.g., editing cassette and nuclease) are under the control of inducible promoters, conditions are provided to induce editing. If none of the components of the editing machinery are under the control of an inducible promoter, editing may proceed immediately after transformation. In certain aspects herein, induction of transcription of one, or, preferably both, of the editing nuclease and editing cassette is prompted by, e.g., using a pL promoter system, where the pL promoter is induced by raising the temperature of the cells in the medium to 42° C. for, e.g., one to many hours to induce expression of the nuclease and editing cassette for cutting and editing. A number of gene regulation control systems have been developed for the controlled expression of genes in plant, microbe, and animal cells, including mammalian cells, including, in addition to the pL promoter, the pPhIF promoter (induced by the addition of 2,4 diacetylphloroglucinol (DAPG)), the pBAD promoter (induced by the addition of arabinose to the cell growth medium), and the rhamnose inducible promoter (induced by the addition of rhamnose to the cell growth medium). Other systems include the tetracycline-controlled transcriptional activation system (Tet-On/Tet-Off, Clontech, Inc. (Palo Alto, Calif.); Bujard and Gossen, PNAS, 89(12):5547-5551 (1992)), the Lac Switch Inducible system (Wyborski et al., Environ Mol Mutagen, 28(4):447-58 (1996); DuCoeur et al., Strategies 5(3):70-72 (1992); U.S. Pat. No. 4,833,080), the ecdysone-inducible gene expression system (No et al., PNAS, 93(8):3346-3351 (1996)), the cumate gene-switch system (Mullick et al., BMC Biotechnology, 6:43 (2006)), and the tamoxifen-inducible gene expression (Zhang et al., Nucleic Acids Research, 24:543-548 (1996)) as well as others.

The present compositions and methods preferably make use of rationally-designed editing cassettes, such as CREATE or CREATE fusion editing cassettes, as described above. Each editing cassette comprises an editing gRNA, and a repair template covalently linked thereto and comprising an intended edit and a PAM or spacer mutation; thus, e.g., a two-cassette multiplex editing cassette comprises a first editing gRNA, a first editing repair template, and a first intended edit and a first PAM or spacer mutation, and at least a second editing gRNA, at least a second repair template, and at least a second intended edit and a second PAM or spacer mutation. In some aspects, a single promoter may drive transcription of both the first and second editing gRNAs and both the first and second repair templates, and in some aspects, separate promoters may drive transcription of the first editing gRNA and first repair template, and transcription of the second editing gRNA and second repair template. In addition, multiplex editing cassettes may comprise nucleic acid elements between the editing cassettes with, e.g., primer sequences, bridging oligonucleotides, and other “cassette-connecting” sequence elements that allow for the assembly of the multiplex editing cassettes.

Once the first round of editing is complete, the cells are allowed to recover and are preferably enriched 214 for cells that have been edited. Enrichment can be performed directly, such as via cells from the population that express a selectable marker, or by using surrogates, e.g., cell surface handles co-introduced with one or more components of the editing components. In certain aspects, cell growth is monitored after editing, and slow growing colonies of cells are identified and selected (e.g., “cherry picked”), or fast-growing colonies are eliminated, resulting in enrichment of edited cells.

At this point in method 200, the cells can be characterized phenotypically or genotypically, or, the cells may undergo additional editing rounds (e.g., some or all of steps 208-214 may be repeated), or, as described below, the cells may proceed to step 216 for curing and further editing.

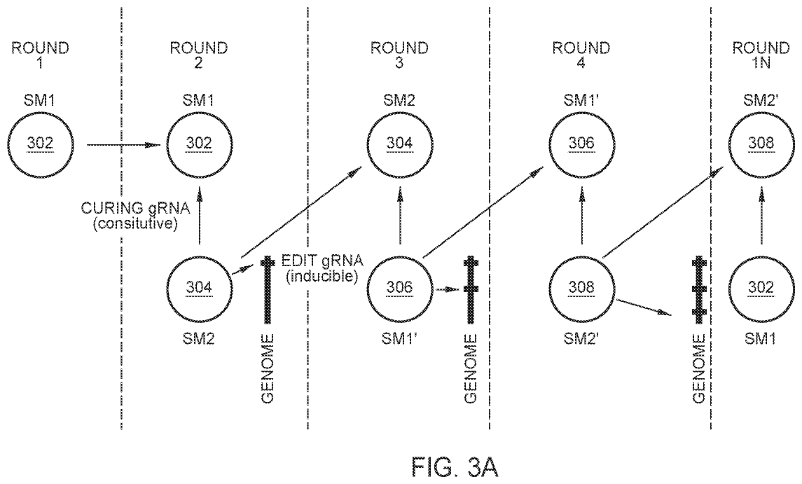

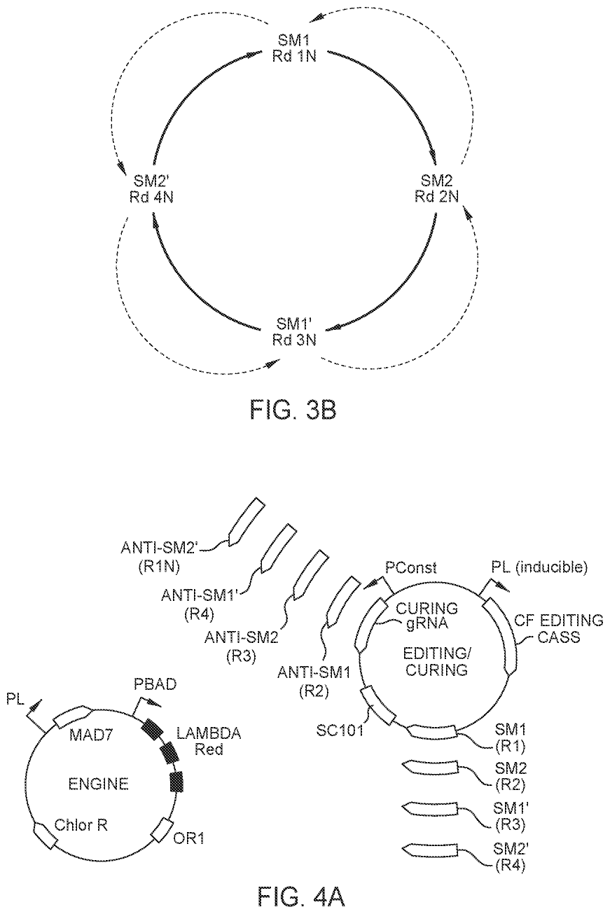

At step 216, a subsequent or successive editing vector is transformed into the cells already having been edited at 212. The subsequent editing vector comprises both an editing cassette and a curing gRNA targeting the initial editing cassette/vector, thus enabling curing of the initial editing cassette from the edited cells, as well as introduction of a second edit into the edited cells. As described in more detail below with reference to FIGS. 3A and 3B, the curing gRNA of the subsequent editing vector is designed to target a sequence disposed on the initial editing vector. In other words, the curing gRNA comprises complementarity to a target curing sequence of the initial editing vectors. In certain aspects, the target curing sequence is comprised of a coding sequence for a selectable marker disposed on the initial editing vector. Generally, the subsequent editing vector 216 is transformed into the edited cells at a transformation efficiency greater than 0.1%, such as a transformation efficiency greater than 1%, 2%, 3%, 4%, 5%, 6%, 7%, 8%, 9%, 10%, or more, to ensure that the proportion of subsequent editing vector to initial editing vector in the cells is large enough to facilitate curing of all or substantially all of the initial editing vector (e.g., prevent the initial editing vector from outcompeting the subsequent editing vector).

Once transformed with the subsequent editing vector 216, the cells are allowed to recover (e.g., grow) and selection is optionally performed 218 to select for cells transformed with the subsequent editing vector, which comprises a selectable marker. Selectable markers and selection medium are employed to select for cells that have received the vector backbone. Commonly used selectable markers include drug selectable markers such as ampicillin/carbenicillin, kanamycin, chloramphenicol, erythromycin, tetracycline, gentamicin, bleomycin, streptomycin, puromycin, hygromycin, blasticidin, and GF18.

Thereafter, conditions are provided to facilitate curing of the initial editing vector 220. Again, “providing conditions” includes incubation of the cells in appropriate medium and may also include providing conditions (e.g., temperature conditions) to facilitate, or even induce via an inducible promoter, transcription of the curing gRNA encoded on the subsequent editing vector, as well as the editing nuclease (which may have been co-delivered with the subsequent editing vector, or previously or subsequently introduced into the cells). In certain aspects, the curing gRNA is under the control of a constitute promoter, and so curing may be initiated upon successful transformation of the subsequent editing vector into the cells. Generally, in order to facilitate multiple iterative rounds of editing with high editing efficiencies, curing of any prior editing plasmids in the cells at operation 220 (and any subsequent curing operations) is performed with a curing efficiency of 99% or greater, such as a curing efficiency of 99.9%, 99.99%, 99.999%, or 99.9999% or greater. As described above, such curing efficiencies of 99% or greater are necessary in order to achieve meaningful editing efficiencies beyond three or more rounds of editing.

Once the initial editing vector has been cured 220, conditions are provided to facilitate editing of the cells at 222 to effect an edit encoded by the subsequent editing vector, e.g., on an editing cassette. Here, “providing conditions” may preferably include providing conditions (e.g., temperature conditions) to induce via an inducible promoter, transcription of the editing cassette encoded on the subsequent editing vector, as well as the editing nuclease. In certain aspects herein, induction of transcription of one, or, preferably both, of the editing nuclease and editing cassette is prompted by, e.g., using a pL promoter system, where the pL promoter is induced by raising the temperature of the cells in the medium to 42° C. for, e.g., one to many hours to induce expression of the nuclease and editing cassette for cutting and editing. However, other gene regulation components or systems are further contemplated.

Once the second round of editing is complete, the cells are allowed to recover and are preferably enriched 224 for cells that have been edited. In certain aspects, to enrich for edited cells, cell growth is monitored after editing, and slow growing colonies of cells are identified and selected (e.g., “cherry picked”), or fast-growing colonies are eliminated.

At this point in method 200, the edited cells can be characterized phenotypically or genotypically, or, the cells may undergo additional curing and editing rounds 126, wherein one or more of steps 216-224 are repeated 228 to effect and stack additional edits in the cells.

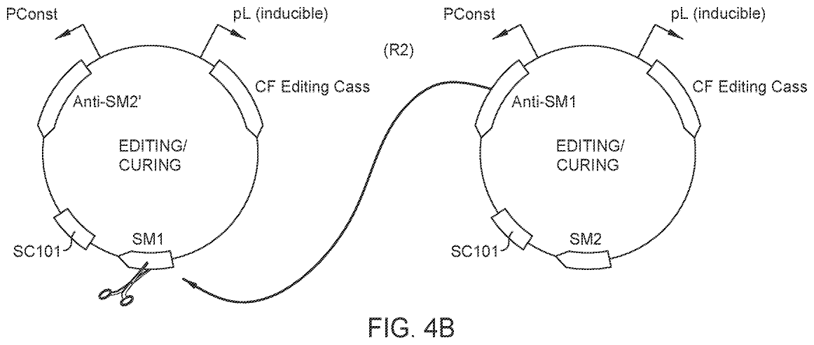

FIGS. 3A and 3B depict an example of an iterative method 200 for editing and curing to effect multiple edits in a population of cells, according the aspects of the present disclosure. Method 200 may be utilized with e.g., the examples of engine and editing vectors depicted in FIGS. 4A-4B, described in more detail below.

Referring now to FIG. 3A, method 200 includes a plurality of iterative editing rounds, such as two, three, four, five, six, seven, eight, nine, or ten or more rounds of editing. For purposes of illustration, five rounds of editing/curing (rounds 1, 2, 3, 4, and 1N, top) are schematically depicted, although more or less rounds are contemplated. As shown, an initial first editing vector 302 is transformed into the cells for round 1 of editing, wherein the first editing vector 302 comprises at least a first editing cassette and a first selectable marker (represented as “SM1”). In certain aspects, first editing vector 302 further comprises a curing gRNA, which may be configured to target the selectable marker sequence of another editing vector utilized in method 200, e.g., editing vector 308 described below. After transformation with first editing vector 302, conditions for editing are provided, and a first edit encoded by the first editing cassette of first editing vector 302 is effected in the plurality of cells.

Thereafter, a second editing vector 304 is transformed into the cells for round 2 of editing. Second editing vector 304 comprises a second editing cassette, a first curing gRNA, and a second selectable marker SM2. The first curing gRNA of second editing vector 304 is designed to target the sequence (e.g., a portion thereof) of first selectable marker SM1 of first editing vector 302. Accordingly, conditions for curing are provided, and the first curing gRNA transcribed from the second editing vector guides the nuclease to the first selectable marker SM1 for cutting, thereby curing the cells of the first editing vector. After this first round of curing, conditions for editing by the second gRNA and second repair template (i.e., second editing cassette) are provided, and the second edit encoded by the second editing vector 304 is effected in the plurality of cells.

Once editing with editing vector 304 is completed, a third editing vector 306 is transformed into the cells for round 3 of editing. Third editing vector 306 comprises a third editing cassette, a second curing gRNA designed to target the second selectable marker SM2 of second editing vector 304, and a third selectable marker SM1′. After transformation, conditions for curing are provided, and the second curing gRNA guides the nuclease to the second selectable marker SM2 on the second editing vector for cutting, thus curing the cells of the second editing vector. After curing, conditions for editing by the third gRNA and third repair template (i.e., third editing cassette) are provided, and a third edit encoded by the third editing vector 306 is effected in the plurality of cells.

In certain aspects, SM1′ is a variant, e.g., a sequence variant, of the selectable marker SM1. For example, in aspects where SM1 and SM1′ are antibiotic resistance genes, SM1 and SM1′ may both be codon-optimized variants of the same antibiotic resistance gene. In some aspects, the selectable markers used on the editing vector in all rounds are different, but in some aspects, the selectable markers used only in adjacent rounds are different; that is, the selectable markers used on the first and second editing vectors are different; the selectable markers used on the second and third editing vectors are different; the selectable marker used on the third and fourth editing vectors are different; however, in certain aspects, the selectable markers used on the first and third editing vectors are the same (or variants of one another); the selectable markers used on the second and fourth editing vectors are the same (or variants of one another); the selectable markers used on the first, third and fifth editing vectors are the same (or variants of one another); the selectable markers used on the second, fourth and sixth editing vectors are the same (or variants of one another), and so on. Accordingly, when performing multiple rounds of iterative editing, in certain aspects, only two selective media (for selecting or enriching transformed cells) may be necessary to carry out the entire iterative process as only two selectable markers are used, thus simplifying the complexity and increasing the efficiency of the iterative process.

After round 3 of editing is completed, curing and editing are again repeated, in e.g., round 4, this time with a fourth editing vector 308. Fourth editing vector 308 comprises a fourth editing cassette, a third curing gRNA designed to target the third selectable marker SM1′ of prior editing vector 306, and a fourth selectable marker SM2′. Similar to SM1′, SM2′ may be a variant SM2, e.g., encoding the same antibiotic resistance gene but having a difference sequence. Curing and editing conditions are sequentially provided to cure the cells of the third editing vector 306 and effect a fourth edit in the plurality of cells. At this point, the method 200 may be repeated (e.g., to round 1N and so on) to effect additional edits in the cells by re-introducing the first editing vector 302, which, may comprise a curing gRNA designed to target SM2′ of editing vector 308′.

Referring now to FIG. 3B, the example of an iterative method 200 lends itself to being a circular process, wherein the same editing components (here, the curing gRNAs and selectable markers) may be re-utilized in multiple iterations to effect a plurality of diverse edits in the cells. Additionally, the transformation of each subsequent editing vector not only facilitates the incorporation of a desired new edit (represented in FIG. 2B as a solid arrow), but further facilitates the curing of an editing vector utilized in the prior editing round (represented in FIG. 2B as a dashed arrow). The system is therefore simultaneously forward- and reverse-acting. As such, the systems and methods described herein not only enable improved editing efficiencies as a result of the targeted curing mechanisms described herein, but do so while facilitating reduced process complexity, since the systems and methods described herein require consideration of fewer design parameters, as well as fewer physical reagents.

FIGS. 4A and 4B depict examples of plasmid architectures for curing of prior editing vectors and effecting subsequent edits utilizing the methods of FIGS. 2 and 3A-3B, according the aspects of the present disclosure. As shown in FIG. 4A, the examples of plasmid architectures comprise an engine vector (left) and an editing vector (right). In this example, the editing vector is configured to 1) cure the cells of a prior editing vector (schematically illustrated in FIG. 4B); and 2) edit the cells.

As shown, the engine vector encodes a nuclease, e.g., MAD7, which is under the control of an inducible or constitute promoter (here, an inducible pL promoter), an origin of replication (e.g., a pUC origin of replication), as well as a selectable marker, such as an antibiotic resistance gene (here, a chloramphenicol resistance gene). In certain aspects, the promoter driving transcription of the nuclease is the same as that driving transcription of an editing cassette and/or curing gRNA on a corresponding editing vector. In certain other aspects, the promoter driving transcription of the nuclease is different than that driving transcription of the editing cassette and/or curing gRNA.

The engine vector further comprises a lambda (λ) Red recombineering system driven by an inducible promoter. The λ Red recombineering system works as a “band aid” or repair system for double-strand breaks in bacteria, and in some species of bacteria the X Red recombineering system (or some other recombineering system) must be present for the double-strand breaks that occur during editing to resolve. In, for example, yeast and other eukaryotic cells, however, recombineering systems are not required. The inducible promoter (in this case pBAD, but other inducible promoters may be used) driving transcription of the λ Red recombineering system components is most preferably a different inducible promoter than that driving transcription of the nuclease and/or the editing cassette, as it is preferred that the recombineering system be active before the nuclease is induced. That is, it is preferred that the “band aid” double-strand break repair machinery be active before the nuclease starts cutting the cellular genome. The resulting edited cells may then be cured according to the methods described herein, and further exposed to additional rounds of editing to “stack” multiple edits with reduced risk of interference or competition for newly-introduced editing vectors from editing vectors introduced in prior editing rounds.

The editing vector on the right in FIG. 4A comprises an inducible promoter (e.g., an inducible pL promoter) driving transcription of an editing cassette, e.g., a CF editing cassette, where the editing cassette includes a coding sequence for an editing gRNA and a repair template (e.g., repair template having a homology arm or “HA”). The repair template—in addition to a sequence for a desired edit in a nucleic acid sequence endogenous to the cell (e.g., the cellular target sequence)—often further comprises a PAM-altering sequence, which is most often a sequence that disables the PAM at the cellular target sequence in the genome.

The editing vector further comprises a promoter (typically a constitutive promoter) driving transcription of a selectable marker, such as an antibiotic resistance gene (e.g., kanamycin or chloramphenicol), followed by an origin of replication (here an SC101 origin, which may be temperature sensitive, but need not be). In certain aspects, all editing vectors designed for/utilized in an iterative editing process (e.g., the vectors described in FIGS. 3A-3B) may comprise the same origin or replication, thereby ensuring that prior (earlier-introduced) vectors do not out-compete subsequent (later-introduced) vectors as a result of replication origin efficiency. In other words, by utilizing editing vectors having the same origin of replication, selective pressure may be applied to the cells to ensure a prior editing vector is cured by the curing gRNA in a subsequent vector.

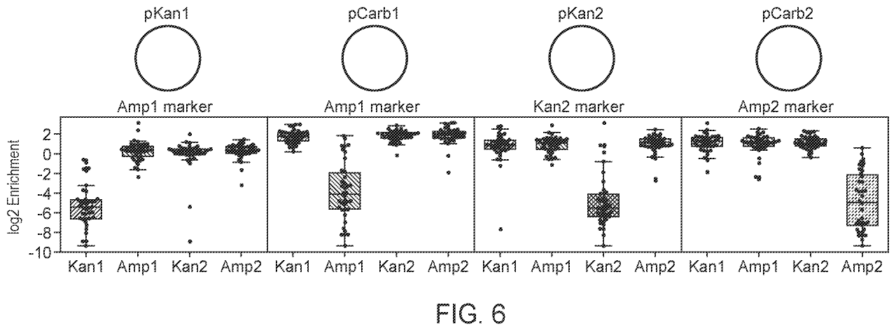

Returning now to FIG. 4A, the editing vector on the right comprises a curing gRNA driven by a constitutive promoter. The curing gRNA is designed to target the selectable marker found in a previously-transformed editing vector during an iterative editing process, as described with reference to FIGS. 2 and 3A-3B, thus facilitating curing of the cells of said previously-transformed editing vector. Accordingly, in any given iterative editing process, the curing gRNA of an editing vector may include one of several curing gRNA designs for targeting a selectable marker of another editing vector utilized during the same iterative editing process. Here, the curing gRNAs include one of an anti-SM1, anti-SM2, anti-SM1′, or anti-SM2′ design, for targeting corresponding selectable markers SM1, SM2, SM1′, or SM2′, as described with reference to the iterative editing process of FIGS. 3A-3B. For illustrative purposes, the corresponding editing rounds described in FIGS. 3A-3B are listed next to each curing gRNA and selective marker in 4A. In certain aspects, optimal gRNAs for targeting each selectable marker may be designed and/or empirically established utilizing gRNA depletion studies (e.g., as demonstrated in FIG. 6). Such experiments measure the activity of every gRNA designed for each selectable marker, and thus, may be used to select gRNA sequences with the greatest depletion activity for implementation in a final editing/curing system.

FIG. 4B depicts the curing mechanism of the editing vector in FIG. 4A. As shown, during a later round of editing (here, round 2 of the iterative editing process of FIGS. 3A-3B), “editing vector 2” is transformed into a plurality of cells already edited by “editing vector 1.” Editing vector 1 comprises a selectable marker SM1, and editing vector 2 comprises a curing gRNA under the control of a constitutive promoter and configured to target SM1 (“anti-SM1”), as well as an editing cassette and a selectable marker SM2. Upon transformation of editing vector 2, its curing gRNA is transcribed and guides a nuclease, which may have been co-introduced into the cells along with editing vector 1 or editing vector 2, to cut or cleave SM1, thereby curing the cells of editing vector 1. Thereafter, effecting a second edit in the cells may be facilitated, e.g., by providing inducing conditions to drive transcription of a CF editing cassette encoded on editing vector 2. Accordingly, editing with editing vector 2 proceeds without competition from editing vector 1, facilitating improved editing efficiency. And, once editing with editing vector 2 is complete, another editing vector having a curing gRNA configured to target SM2 may be transformed into the cells, and the curing/editing process repeated.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Iterative Curing

### Example 1: Evaluating Libraries of sgRNAs for Effectiveness

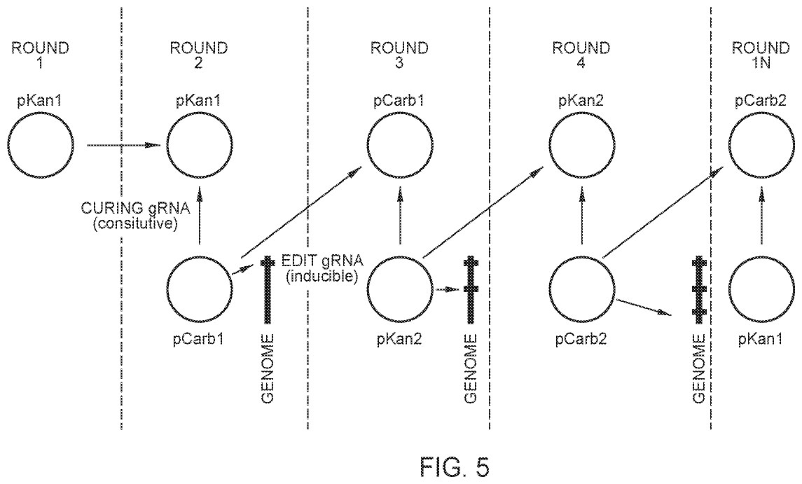

An iterative curing system architecture was designed for MG1655 cells as shown in FIG. 5. In particular, plasmids were assembled with a selectable marker picked from one of two sets of codon-optimized antibiotic resistance gene pairs, e.g., “Kan1” or “Kan2” or “Carb1” or “Carb2”. The selectable markers in this experimental protocol were utilized as the target curing sites (i.e., “target markers”) for curing gRNAs. For each codon-optimized antibiotic resistance gene, all spacer sites for targeting and cutting the antibiotic resistance gene with a MAD7 nuclease were identified and mapped (for both forward and reverse DNA strands), and corresponding curing gRNAs were designed. The curing gRNAs were then empirically evaluated for their effectiveness in targeting the corresponding target marker for cutting by the nuclease.

To evaluate the effectiveness of the curing gRNAs, gRNA libraries for each target marker were designed, synthesized, and cloned into plasmid backbones for each antibiotic selectable marker. The libraries were then tested for their ability to self-cure in the presence of the corresponding target marker. In other words, the curing gRNAs were tested for enrichment or depletion when exposed to their target marker. Each library was co-transformed into separate cell sample isolates with an engine plasmid encoding a MAD7 nuclease and the corresponding target marker, and, after provision of curing conditions, the curing gRNA content for each cell sample was measured, as shown in FIG. 6. Those curing gRNAs exhibiting the lowest counts (e.g., the highest activity, thereby causing depletion thereof) in each sample were then selected for further characterization.

gRNA activity assays were performed by transforming 100 ng of purified plasmid DNA (composed of a comprehensive gRNA library in each of the 4 antibiotic selectable marker containing vectors) into 30 μL of competent cells in 1 cm cuvettes by electroporation at 1800 mV. Competent cells were made from the MG1655 E. coli strain containing the MAD7 nuclease. Following transformation, the cells were recovered in 500 μL of SOB media for 1 hour, and then diluted into 25 mL of SOB containing the appropriate antibiotic (100 μg/mL for the carbenicillin resistant plasmids and 50 μg/mL for the kanamycin resistant plasmids). Following outgrowth for 24 hours, plasmid DNA was purified from cell populations. Plasmid DNA samples were amplified by PCR to add sequencing adapters and sample indices, and then sequenced in an Illumina MiSeq sequencer using a 2×100 bp sequencing kit. Frequency of each cassette before and after propagation in the presence of MAD7 was then used to determine the relative activity of each gRNA design for self-curing/cleavage activity. All analyses were done using a combination of USEARCH to tally sequence abundance and Python-based algorithms to calculate frequency and enrichment of each gRNA in the population. Again, the resulting analysis is depicted in FIG. 6.

### Example 2: Characterizing Selected gRNAs for Curing Efficiency

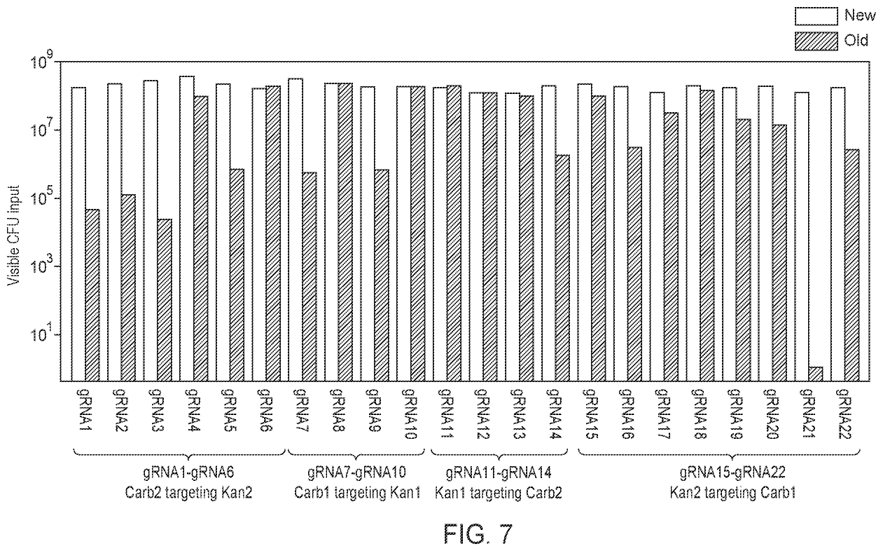

The selected curing gRNAs were further individually characterized by curing efficiency to pick the single-best curing gRNA for each target marker. To do this, each curing gRNA was assembled into a plasmid with a “new” selectable marker, and, along with an engine plasmid encoding a MAD7 nuclease and its own Chlor selectable marker, introduced into cells already transformed with a plasmid carrying a corresponding target marker, or “old” marker. Again, the same codon-optimized antibiotic resistance genes were used as selective markers (Carb1, Carb2, Kan1, and Kan2). Saturated liquid outgrowth comprising the transformed cells was diluted 40× in liquid media selective for either the old or the new marker, and grown at 30° C. for 6 hours, with shaking. Viable cell counts (e.g., CFUs) for each antibiotic were measured using the start growth time method (see, R Hazan, Y.-A. Que, D. Maura, L. G. Rahme, A method for high throughput determination of viable bacteria cell counts in 96-well plates. BMC Microbiol. 12, 259 (2012)). The results are depicted in FIG. 7.

FIG. 7 depicts the measured colony-forming units for each cell isolate on media selective for the old marker (bars with hatching, “old media”) and media selective for the new marker (bars with no hatching, “new media”). Cells that exhibited substantial growth on the new media, but did not grow well on the old media, indicated a high curing efficiency of the corresponding gRNA transformed therein. In other words, the old marker was successfully cured from these cells, while the new marker was still present therein. On the other hand, cells that exhibited substantial growth on media selective for the old marker indicated low curing efficiency of the corresponding gRNA, as the old marker was not successfully cured and was still present in these cells. Curing efficiency in each case can be computed by the following equation:

curing efficiency=CFU_{new}/CFU_{old}.

### Example 3: Targeting Versus Non-Targeting gRNAs

To confirm that a curing gRNA targeting the “old” plasmid was required in order to achieve high curing efficiency thereof, and that the curing efficiencies observed in other studies were not simply caused by dilution or competition of origins, curing efficiencies of target and non-target plasmids were compared for the previously selected gRNAs. Cell isolates were first transformed with a plasmid carrying a target selectable marker, and were later transformed with an engine plasmid encoding a MAD7 nuclease and either an active gRNA designed to target the selectable marker (the “right gRNA”), or a non-targeting gRNA (a “wrong gRNA”). The cell isolates were plated on media selective for the old plasmid and incubated at either 30° C. (non-inducing conditions) or 42° C. (inducing conditions), diluted 40×, and then incubated again on selective media at 30° C. and counted.

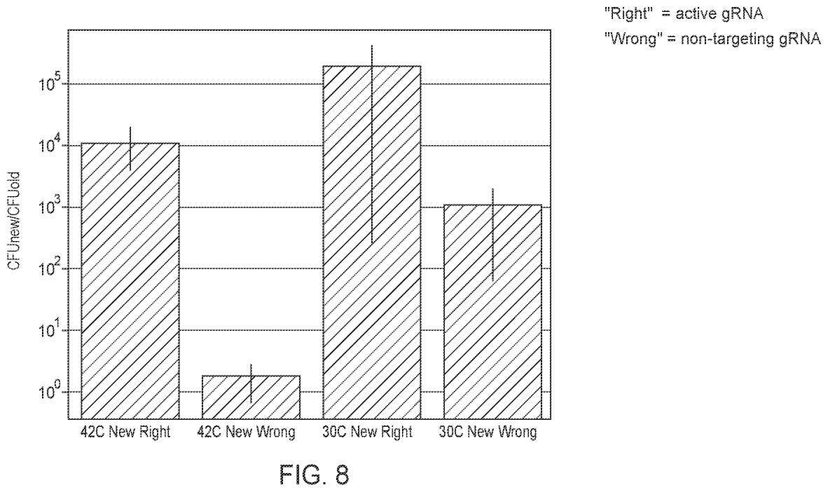

As shown in FIG. 8, for cells incubated in non-inducing conditions, there was over 100-fold less colonies observed in cells that were transformed with a targeting gRNA as compared to cells transformed with a non-targeting gRNA. There was an even greater difference in cell growth observed in cells incubated in inducing conditions. This indicates that utilization of a curing gRNA specifically targeting the “old” plasmid is required in order to achieve high curing efficiency thereof.

### Example 4: Effect of DNA Concentration on Curing Efficiency

To determine the effect of DNA concentration used during transformation on curing efficiency, four plasmids were designed according to the architectures illustrated in FIG. 5, and three successive rounds of transformation/curing (P1, P2, P3) were performed in cell sample isolates utilizing five different concentrations (2.7 ng, 27 ng, 54 ng, 108 ng, 0 ng) of each subsequently-introduced (“new”) plasmid. Again, each new plasmid comprised a curing gRNA configured to target a selectable marker of the previously-introduced (“old”) plasmid. After round 3 (P3) of transformation/curing, the cell samples were analyzed via qPCR to determine the concentration of each plasmid therein.

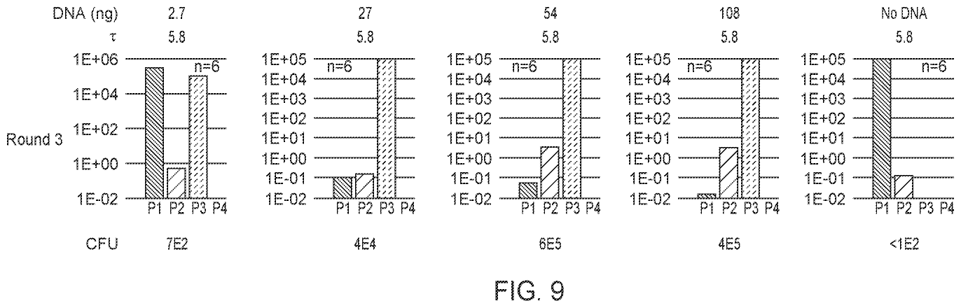

As shown in FIG. 9, when only 2.7 ng of each new plasmid was transformed in each successive curing round, a sizeable quantity of the first (left-most bar) and second (middle bar) plasmids remained uncured, as the old plasmids were able to out-compete (e.g., via replication) each new plasmid. However, when utilizing 27 ng or more of each new plasmid during transformation, a high curing efficiency of the old plasmids was achieved, with at least 5-fold greater curing efficiency of the first plasmid as compared with, e.g., the 2.7 ng sample.

### Example 5: Iterative Rounds of High Fidelity Curing on Pooled Samples

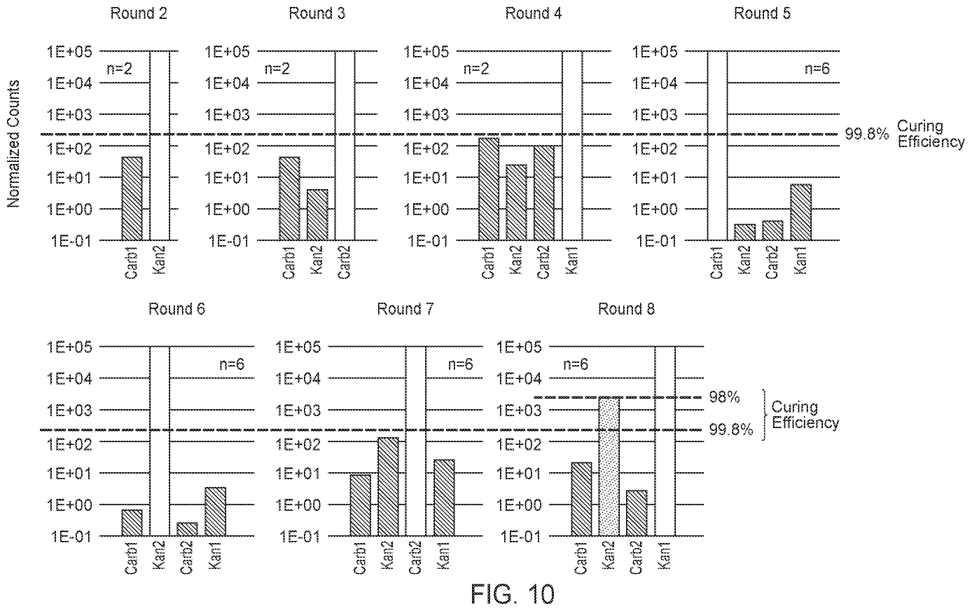

To test the effectiveness of the curing systems described herein at library-scale and over multiple (>4) iterations, four plasmids were designed according to the architectures illustrated in FIG. 5 for successive rounds of transformation/curing with a population of cells. Accordingly, each plasmid comprised a curing gRNA configured to target a selective marker (e.g., Carb1, Kan2, Carb2, or Kan1) of another plasmid transformed into the cells in a prior round of curing. After each round of transformation, cells were grown out, cherry picked for slow-growing colonies, and then repooled for another round of transformation. The abundance of each plasmid in the cells was measured after each transformation round via qPCR, and the results are depicted in FIG. 10.

As shown, a 99.9% curing efficiency of the previously-introduced, “old” plasmid (bars with hatching), as compared to the recently-introduced, “new” plasmid (bars without hatching), was achieved for at least 7 rounds of transformation/curing in the cell population. During the 8′ round of curing, the curing efficiency of the old plasmid decreased to 98%, which may indicate that cell strains needed to be isolated prior to this transformation/curing round in order to proceed to further iterations.

While previous methods have enabled 2-3 rounds of successful plasmid curing, FIG. 10 illustrates the first successful demonstration known to the inventors of the present disclosure of iterative, high efficiency curing (>99%) beyond three rounds. Furthermore, the results in FIG. 10 demonstrate that such high-efficiency curing is possible in pooled cell samples, thus demonstrating viability at a library scale, whereas previous curing methods have only been demonstrated with cell sample isolates. Accordingly, the inventors of the present disclosure have demonstrated a defined and iterative curing process to enable several rounds of editing to be performed reliably and in rapid succession using genome-scale libraries of edits.

### Example 6: Iterative Curing Enables Rapid Strain Improvement Over Multiple Rounds

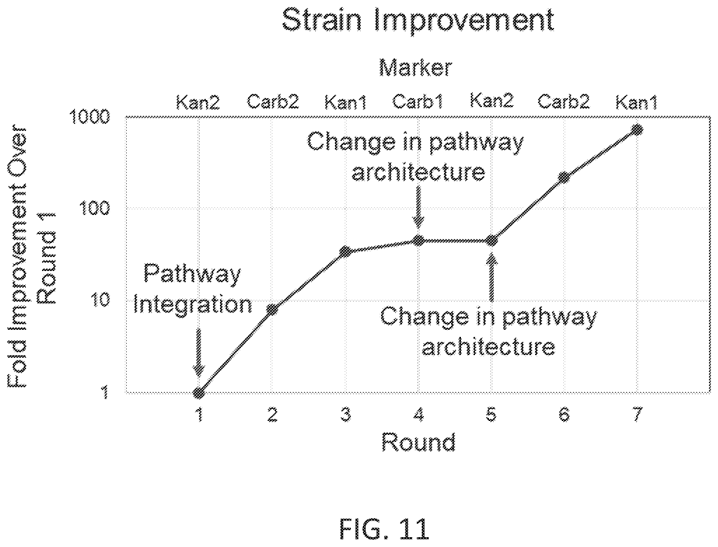

The iterative curing technology is applied to rapidly increase the production of a proprietary molecule in E. coli by over 750 fold in seven rounds of iterative editing, as illustrated by FIG. 11. The pathway driving the production of the molecule is integrated into the genome in round one using a plasmid containing a Kan2 marker. Iterative editing for six subsequent rounds is enabled via curing the previous plasmids Carb2, Kan1, and Carb1 plasmids, in that order. Curing enabled library scale (e.g. more than 1000 designs) editing in rounds two, three, six, and seven, as well as the integration of large inserts (e.g. >1 kb) in rounds one and four.

### Example 7: Strain Improvement During Round Six of Iterative Editing

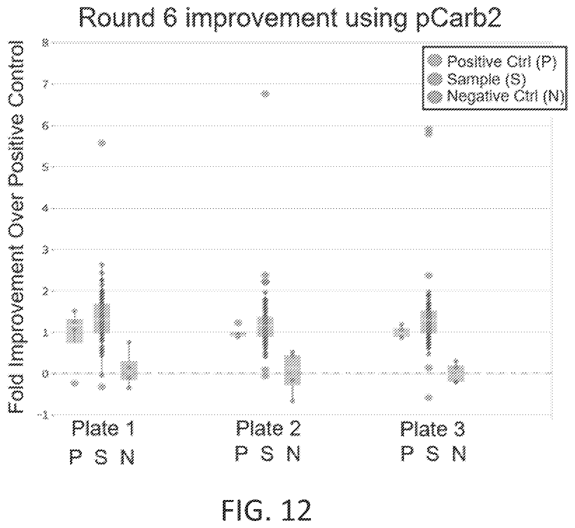

Fold improvement (FI) of samples edited in round six compared to the positive control (top producer from the previous round five) is shown in FIG. 12. FI for round six samples vary widely from 6.8 to −0.5 and over 40% of samples have significantly higher or lower FI values compared to the positive control. Samples with similar FI as the positive control are either not edited or edited with neutral mutations. The high number of samples edited with significantly different FI values than the positive control demonstrates the effectiveness of curing previous plasmids (in rounds one to five), since the editing efficiency would have been less than 3% or 10% with a curing efficiency of 90% or 99%, respectively, during each prior round as shown in FIG. 1A.

While this invention is satisfied by aspects in many different forms, as described in detail in connection with preferred aspects of the invention, it is understood that the present disclosure is to be considered as an example of the principles of the invention and is not intended to limit the invention to the specific aspects illustrated and described herein. Numerous variations may be made by persons skilled in the art without departure from the spirit of the invention. The scope of the invention will be measured by the appended claims and their equivalents. The abstract and the title are snot to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

A variety of further modifications and improvements in and to the compositions, methods, and modified cells of the present disclosure will be apparent to those skilled in the art. The following non-limiting, embodiments are specifically envisioned:

1. A method for iterative nucleic acid-guided nuclease editing, comprising:
providing a plurality of edited cells, the edited cells modified during an initial round of editing to include a first genomic edit, the edited cells comprising:
a first editing vector comprising a first editing cassette and a first selectable marker;
transforming the cells with a second editing vector, the second editing vector comprising:
a second editing cassette, a second selectable marker, and a first nucleic acid sequence coding for a first curing gRNA configured to target the first selectable marker of the first editing vector;
providing conditions for curing the first editing vector in the plurality of edited cells, wherein curing the first editing vector comprises cleaving the first editing vector at the first selectable marker with a nucleic acid-guided nuclease guided by the first curing gRNA; and,
providing conditions for editing the plurality of edited cells with the second editing cassette, wherein the second editing cassette is configured to further modify the edited cells to include a second genomic edit.

2. The method of embodiment 1, wherein the first editing vector and the second editing vector comprise the same type of origin of replication.

3. The method of embodiment 1, wherein the first selectable marker and the second selectable marker comprise antibiotic resistance genes.

4. The method of embodiment 1, wherein the first curing gRNA is under the control of a constitutive promoter.

5. The method of embodiment 1, wherein the first editing cassette and/or the second editing cassette are under the control of an inducible promoter.

6. The method of embodiment 1, wherein the first editing cassette comprises a first nucleic acid sequence coding for a first editing gRNA covalently linked to a repair template for effecting the first edit.

7. The method of embodiment 6, wherein the second editing cassette comprises a second nucleic acid sequence coding for a second editing gRNA covalently linked to a repair template for effecting the second edit.

8. The method of embodiment 1, further comprising:
transforming the edited cells with the nucleic acid-guided nuclease or a sequence coding for the nucleic acid-guided nuclease.

9. The method of embodiment 8, wherein the nuclease includes a MAD-series nuclease, nickase, or variant thereof.

10. The method of embodiment 1, wherein the first editing vector is cured with a curing efficiency of 99.9%/6 or greater.

11. The method of embodiment 1, further comprising:
transforming the cells with a third editing vector, the third editing vector comprising:
a third editing cassette, a third selectable marker, and a second nucleic acid sequence coding for a second curing gRNA configured to target the second selectable marker of the second editing vector;
providing conditions for curing the second editing vector in the plurality of edited cells, wherein curing the second editing vector comprises cleaving the second editing vector at the second selectable marker with the nucleic acid-guided nuclease guided by the second curing gRNA; and,
providing conditions for editing the plurality of edited cells with the third editing cassette, wherein the third editing cassette is configured to further modify the edited cells to include a third genomic edit.

12. The method of embodiment 11, wherein the second editing vector is cured with a curing efficiency of 99.9% or greater.

13. The method of embodiment 11, further comprising:
transforming the cells with a fourth editing vector, the fourth editing vector comprising:
a fourth editing cassette, a fourth selectable marker, and a third nucleic acid sequence cording for a third curing gRNA configured to target the third selectable marker of the third editing vector;
providing conditions for curing the third editing vector in the plurality of edited cells, wherein curing the third editing vector comprises cleaving the third editing vector at the third selectable marker with the nucleic acid-guided nuclease guided by the third curing gRNA; and,
providing conditions for editing the plurality of edited cells with the fourth editing cassette, wherein the fourth editing cassette is configured to further modify the edited cells to include a fourth genomic edit.

14. The method of embodiment 13, wherein the third editing vector is cured with a curing efficiency of 99.9% or greater.

15. A method for iterative nucleic acid-guided nuclease editing, comprising:
providing a plurality of edited cells, the edited cells modified during an initial round of editing to include a first genomic edit, the edited cells comprising:
a first editing vector comprising a first editing cassette and a first selectable marker;
transforming the cells with a second editing vector, the second editing vector comprising:
a second editing cassette, a second selectable marker, and a first nucleic acid sequence coding for a first curing gRNA configured to target the first selectable marker of the first editing vector;
providing conditions for curing the first editing vector in the plurality of edited cells with a curing efficiency of at least 99%, wherein curing the first editing vector comprises cleaving the first editing vector at the first selectable marker with a nucleic acid-guided nuclease guided by the first curing gRNA;
providing conditions for editing the plurality of edited cells with the second editing cassette, wherein the second editing cassette is configured to further modify the edited cells to include a second genomic edit;
transforming the cells with a third editing vector, the third editing vector comprising: a third editing cassette, a third selectable marker, and a second nucleic acid sequence coding for a second curing gRNA configured to target the second selectable marker of the second editing vector;
providing conditions for curing the second editing vector in the plurality of edited cells with a curing efficiency of at least 99%, wherein curing the second editing vector comprises cleaving the second editing vector at the second selectable marker with the nucleic acid-guided nuclease guided by the second curing gRNA; and,
providing conditions for editing the plurality of edited cells with the third editing cassette, wherein the third editing cassette is configured to further modify the edited cells to include a third genomic edit.

16. The method of embodiment 15, further comprising:
transforming the cells with a fourth editing vector, the fourth editing vector comprising: a fourth editing cassette, a fourth selectable marker, and a third nucleic acid sequence coding for a third curing gRNA configured to target the third selectable marker of the third editing vector;
providing conditions for curing the third editing vector in the plurality of edited cells with a curing efficiency of at least 99%, wherein curing the third editing vector comprises cleaving the third editing vector at the third selectable marker with the nucleic acid-guided nuclease guided by the third curing gRNA; and,
providing conditions for editing the plurality of edited cells with the fourth editing cassette, wherein the fourth editing cassette is configured to further modify the edited cells to include a fourth genomic edit.

17. The method of embodiment 15, wherein the first editing vector and the second editing vector comprise the same type of origin of replication.

18. The method of embodiment 15, wherein the first selectable marker and the second selectable marker comprise antibiotic resistance genes.

19. A method for iterative nucleic acid-guided nuclease editing, comprising:
providing a plurality of edited cells, the edited cells modified during an initial round of editing to include a plurality of first genomic edits, the edited cells comprising:
   a plurality of first editing vectors, each vector of the plurality of first editing vectors comprising:
at least one editing cassette of a library of first editing cassettes, wherein one or more editing cassettes of the library of first editing cassettes comprises a unique edit of the plurality of first genomic edits; and
a first selectable marker;
transforming the cells with a plurality of second editing vectors, each vector of the second editing vectors comprising:
at least one editing cassette of a library of second editing cassettes, wherein one or more editing cassettes of the library of second editing cassettes comprises a unique edit of a plurality of second genomic edits;
a second selectable marker; and
a first nucleic acid sequence coding for a first curing gRNA configured to target the first selectable marker of the plurality of first editing vectors;
providing conditions for curing the plurality of first editing vectors in the plurality of edited cells, wherein curing the plurality of first editing vectors comprises cleaving the plurality of first editing vectors at the first selectable marker with a nucleic acid-guided nuclease guided by the first curing gRNA of the plurality of second editing vectors; and,
providing conditions for editing the plurality of edited cells with the plurality of second editing vectors, wherein the plurality of second editing vectors are configured to further modify the edited cells to include the plurality of second genomic edits.

20. The method of embodiment 19, wherein the plurality of first editing vectors is cured with a curing efficiency of 99.9% or greater.

1. A method for iterative nucleic acid-guided nuclease editing, comprising:
providing a plurality of edited cells, the edited cells modified during an initial round of editing to include a first genomic edit, the edited cells comprising:
a first editing vector comprising a first editing cassette and a first selectable marker;
transforming the cells with a second editing vector, the second editing vector comprising:
a second editing cassette, a second selectable marker, and a first nucleic acid sequence coding for a first curing gRNA configured to target the first selectable marker of the first editing vector;
providing conditions for curing the first editing vector in the plurality of edited cells, wherein curing the first editing vector comprises cleaving the first editing vector at the first selectable marker with a nucleic acid-guided nuclease guided by the first curing gRNA; and,
providing conditions for editing the plurality of edited cells with the second editing cassette, wherein the second editing cassette is configured to further modify the edited cells to include a second genomic edit.

2. The method of claim 1, wherein the first editing vector and the second editing vector comprise the same type of origin of replication.

3. The method of claim 1, wherein the first selectable marker and the second selectable marker comprise antibiotic resistance genes.

4. The method of claim 1, wherein the first curing gRNA is under the control of a constitutive promoter.

5. The method of claim 1, wherein the first editing cassette and/or the second editing cassette are under the control of an inducible promoter.

6. The method of claim 1, wherein the first editing cassette comprises a first nucleic acid sequence coding for a first editing gRNA covalently linked to a repair template for effecting the first edit.

7. The method of claim 6, wherein the second editing cassette comprises a second nucleic acid sequence coding for a second editing gRNA covalently linked to a repair template for effecting the second edit.

8. The method of claim 1, further comprising:
transforming the edited cells with the nucleic acid-guided nuclease or a sequence coding for the nucleic acid-guided nuclease.

9. The method of claim 8, wherein the nuclease includes a MAD-series nuclease, nickase, or variant thereof.

10. The method of claim 1, wherein the first editing vector is cured with a curing efficiency of 99.9% or greater.

11. The method of claim 1, further comprising:
transforming the cells with a third editing vector, the third editing vector comprising:
a third editing cassette, a third selectable marker, and a second nucleic acid sequence coding for a second curing gRNA configured to target the second selectable marker of the second editing vector;
providing conditions for curing the second editing vector in the plurality of edited cells, wherein curing the second editing vector comprises cleaving the second editing vector at the second selectable marker with the nucleic acid-guided nuclease guided by the second curing gRNA; and,
providing conditions for editing the plurality of edited cells with the third editing cassette, wherein the third editing cassette is configured to further modify the edited cells to include a third genomic edit.

12. The method of claim 11, wherein the second editing vector is cured with a curing efficiency of 99.9% or greater.

13. The method of claim 11, further comprising:
transforming the cells with a fourth editing vector, the fourth editing vector comprising:
a fourth editing cassette, a fourth selectable marker, and a third nucleic acid sequence cording for a third curing gRNA configured to target the third selectable marker of the third editing vector;
providing conditions for curing the third editing vector in the plurality of edited cells, wherein curing the third editing vector comprises cleaving the third editing vector at the third selectable marker with the nucleic acid-guided nuclease guided by the third curing gRNA; and,
providing conditions for editing the plurality of edited cells with the fourth editing cassette, wherein the fourth editing cassette is configured to further modify the edited cells to include a fourth genomic edit.

14. The method of claim 13, wherein the third editing vector is cured with a curing efficiency of 99.9% or greater.

15. A method for iterative nucleic acid-guided nuclease editing, comprising:
providing a plurality of edited cells, the edited cells modified during an initial round of editing to include a first genomic edit, the edited cells comprising:
a first editing vector comprising a first editing cassette and a first selectable marker;
transforming the cells with a second editing vector, the second editing vector comprising:
a second editing cassette, a second selectable marker, and a first nucleic acid sequence coding for a first curing gRNA configured to target the first selectable marker of the first editing vector;
providing conditions for curing the first editing vector in the plurality of edited cells with a curing efficiency of at least 99%, wherein curing the first editing vector comprises cleaving the first editing vector at the first selectable marker with a nucleic acid-guided nuclease guided by the first curing gRNA;
providing conditions for editing the plurality of edited cells with the second editing cassette, wherein the second editing cassette is configured to further modify the edited cells to include a second genomic edit;
transforming the cells with a third editing vector, the third editing vector comprising: a third editing cassette, a third selectable marker, and a second nucleic acid sequence coding for a second curing gRNA configured to target the second selectable marker of the second editing vector;
providing conditions for curing the second editing vector in the plurality of edited cells with a curing efficiency of at least 99%, wherein curing the second editing vector comprises cleaving the second editing vector at the second selectable marker with the nucleic acid-guided nuclease guided by the second curing gRNA; and,
providing conditions for editing the plurality of edited cells with the third editing cassette, wherein the third editing cassette is configured to further modify the edited cells to include a third genomic edit.

16. The method of claim 15, further comprising:
transforming the cells with a fourth editing vector, the fourth editing vector comprising: a fourth editing cassette, a fourth selectable marker, and a third nucleic acid sequence coding for a third curing gRNA configured to target the third selectable marker of the third editing vector;
providing conditions for curing the third editing vector in the plurality of edited cells with a curing efficiency of at least 99%, wherein curing the third editing vector comprises cleaving the third editing vector at the third selectable marker with the nucleic acid-guided nuclease guided by the third curing gRNA; and,
providing conditions for editing the plurality of edited cells with the fourth editing cassette, wherein the fourth editing cassette is configured to further modify the edited cells to include a fourth genomic edit.

17. The method of claim 15, wherein the first editing vector and the second editing vector comprise the same type of origin of replication.

18. The method of claim 15, wherein the first selectable marker and the second selectable marker comprise antibiotic resistance genes.

19. A method for iterative nucleic acid-guided nuclease editing, comprising:
providing a plurality of edited cells, the edited cells modified during an initial round of editing to include a plurality of first genomic edits, the edited cells comprising:
a plurality of first editing vectors, each vector of the plurality of first editing vectors comprising:
at least one editing cassette of a library of first editing cassettes, wherein one or more editing cassettes of the library of first editing cassettes comprises a unique edit of the plurality of first genomic edits; and
a first selectable marker;
transforming the cells with a plurality of second editing vectors, each vector of the second editing vectors comprising:
at least one editing cassette of a library of second editing cassettes, wherein one or more editing cassettes of the library of second editing cassettes comprises a unique edit of a plurality of second genomic edits;
a second selectable marker; and
a first nucleic acid sequence coding for a first curing gRNA configured to target the first selectable marker of the plurality of first editing vectors;
providing conditions for curing the plurality of first editing vectors in the plurality of edited cells, wherein curing the plurality of first editing vectors comprises cleaving the plurality of first editing vectors at the first selectable marker with a nucleic acid-guided nuclease guided by the first curing gRNA of the plurality of second editing vectors; and,
providing conditions for editing the plurality of edited cells with the plurality of second editing vectors, wherein the plurality of second editing vectors are configured to further modify the edited cells to include the plurality of second genomic edits.

20. The method of claim 19, wherein the plurality of first editing vectors is cured with a curing efficiency of 99.9% or greater.
